(19) United States
(12) Patent Application Publication
(10) Pub. No.: US 2023/0141283 A1
(43) Pub. Date: May 11, 2023

(54) METABOLIC ENGINEERING OF CUPRIAVIDUS NECATOR FOR IMPROVED FORMATE UTILIZATION

(71) Applicant: Alliance for Sustainable Energy, LLC, Golden, CO (US)

(72) Inventors: Christopher Hugh CALVEY, Denver, CO (US); Christopher W. JOHNSON, Denver, CO (US)

(21) Appl. No.: 18/053,696

(22) Filed: Nov. 8, 2022

Related U.S. Application Data
(60) Provisional application No. 63/277,080, filed on Nov. 8, 2021.

(51) Int. Cl. C12N 1/20; C12N 15/10

(52) U.S. Cl. CPC C12N 1/20; C12N 15/102

(57) ABSTRACT

Disclosed herein are compositions and methods to improve Cupriavidus sp. as a host for formate conversion. Also disclosed herein are compositions and methods to improve growth of non-naturally occurring Cupriavidus sp. on carbon dioxide, succinate, formate and fructose as sole carbon sources.

### CROSS-REFERENCE TO RELATED APPLICATIONS

This application claims priority under 35 U.S.C. § 119 to U.S. provisional patent application No. 63/277,080 filed on 8 Nov. 2021, the contents of which are hereby incorporated in their entirety.

### CONTRACTUAL ORIGIN

The United States Government has rights in this invention under Contract No. DE-AC36-08G028308 between the United States Department of Energy and the Alliance for Sustainable Energy, LLC, the Manager and Operator of the National Renewable Energy Laboratory.

### SEQUENCE LISTING

This application contains a Sequence Listing which has been submitted via the Patent Center and is hereby incorporated by reference in its entirety. The XML copy as filed herewith was originally created on 8 Nov. 2022. The XML copy as filed herewith is named NREL 21-62.xml, is 75 kilobytes in size and is submitted with the instant application.

### BACKGROUND

Atmospheric concentrations of carbon dioxide have reached the highest levels present on Earth for several million years and are steadily increasing. In order to avert the catastrophic effects of climate change, global civilization must rapidly deploy technologies capable of reducing emissions of CO₂ and other greenhouse gases toward net zero levels. One strategy entails capturing and converting CO₂ at the point of emission, such as a variety of industrial waste gas streams, where CO₂ is available at a relatively high concentration. Using renewable sources of electricity, electrolysis systems have the potential to electrochemically reduce CO₂ to a multitude of products including carbon monoxide, formate, ethanol, ethylene, and other hydrocarbons.

Highly efficient electrochemical reduction of CO₂ to formate and formic acid has been previously demonstrated. Formic acid is itself a valuable commodity used in various agricultural, chemical, pharmaceutical, and textile industries. Recently, formate has also gathered significant interest as a potential feedstock for microbial upgrading, as it can be consumed as the sole source of carbon and energy by some microbial species, termed formatotrophs. It is also highly water soluble, which enables microbial conversion without the safety, transport, solubility, and mass-transfer challenges associated with gaseous feedstocks. Therefore, it is an ideal intermediate molecule to serve as a bridge between biological and electrochemical conversion technologies. Within a formate bioeconomy, cheap renewable electricity produced at off-peak hours could be used to convert CO₂ to formate, which can be stored, and later converted by metabolically engineered microbes into a virtually limitless spectrum of fuels, chemicals, and materials.

### SUMMARY

In an aspect, disclosed herein is a non-naturally occurring Cupriavidus sp. comprising at least one genetic deletion wherein the at least one genetic deletion improves growth on formate as a sole carbon source by up to 24 percent over a naturally occurring Cupriavidus sp. In an embodiment, the Cupriavidus sp. genotype comprises ΔhoxFUYHWI ΔhypA2B2F2. In an embodiment, the Cupriavidus sp. genotype comprises ΔhoxKGZMLOQRTV ΔhypA1B1F1CDEX ΔhoxABCJ. In an embodiment, the Cupriavidus sp. genotype comprises ΔcbbR′ ΔcbbLpSpXpYpEpFpPpTpZpGpKpAp. In an embodiment, the Cupriavidus sp. genotype comprises ΔpHG1. In an embodiment, the Cupriavidus sp. genotype comprises ΔphcA. In an embodiment, the Cupriavidus sp. genotype comprises ΔpHG1 ΔphcA. In an embodiment, the Cupriavidus sp. grows in minimal salt media supplemented with 50 mM sodium formate at a growth rate of up to 2.18 times greater than a wildtype Cupriavidus sp. grown in minimal salt media supplemented with 50 mM sodium formate. In an embodiment, the Cupriavidus sp. grows in minimal salt media supplemented with 50 mM sodium formate up to a 34 percent greater optical density at 600 nm compared to a wildtype Cupriavidus sp. grown in minimal salt media supplemented with 50 mM sodium formate. In an embodiment, the Cupriavidus sp. is Cupriavidus necator.

In an aspect, disclosed herein is a non-naturally occurring Cupriavidus sp. comprising at least one genetic deletion wherein the at least one genetic deletion improves growth on fructose as a sole carbon source by up to 19 percent over a naturally occurring Cupriavidus sp. In an embodiment, the Cupriavidus sp. genotype comprises ΔpHG1. In an embodiment, the Cupriavidus sp. genotype comprises ΔphcA. In an embodiment, the Cupriavidus sp. genotype comprises ΔpHG1 ΔphcA.

In an aspect, disclosed herein is a non-naturally occurring Cupriavidus sp. comprising at least one genetic deletion wherein the at least one genetic deletion improves growth on succinate as the sole carbon source by up to 7 percent over a naturally occurring Cupriavidus sp. In an embodiment, the Cupriavidus sp. genotype is selected from the group consisting of ΔpHG1 ΔphcA and ΔphcA.

In an aspect, disclosed herein is a non-naturally occurring Cupriavidus sp. comprising at least one genetic deletion wherein the at least one genetic deletion improves growth on carbon dioxide as a sole carbon source when compared to a naturally occurring Cupriavidus sp. In an embodiment, the Cupriavidus sp. genotype comprises a deletion of at least one copy of the CBB operon. In an embodiment, the Cupriavidus sp. genotype comprises a deletion of a CBB operon within a megaplasmid. In an embodiment, the Cupriavidus sp. genotype comprises a deletion of a chromosomal CBB operon.

In an aspect, disclosed herein is a method for deleting a megaplasmid within an organism comprising deleting a gene on the megaplasmid that encodes for a toxin; and further comprising deleting a replication region of the megaplasmid. In an embodiment, the organism is a Cupriavidus sp. In an embodiment, the megaplasmid is pHG1.

Other objects, advantages, and novel features of the present invention will become apparent from the following detailed description of the invention when considered in conjunction with the accompanying drawings.

### DESCRIPTION OF THE DRAWINGS

FIGS. 1a, 1b and 1c depict performance of evolved and engineered strains grown on formate at microplate (200 μL) scale. Growth curves of strains obtained from FIG. 1a) ALE or from FIG. 1b), FIG. 1c) rational metabolic engineering, cultivated on minimal media with 50 mM formate using a Bioscreen C Pro microplate reader. Data shown as average of quadruplicate wells and standard deviations indicated as error bars.

FIGS. 2a and 2b depict cultivation of engineered strains grown on formate or fructose at flask (50 mL) scale for RNA-seq transcriptomics from operons of interest. Transcriptomics was conducted during shake flask cultivation on minimal media with FIG. 2a) 50 mM formate or FIG. 2b) 2 g/L fructose, with data shown as averages of triplicate flasks and standard deviations indicated as error bars. Larger circles outlined in black represent sampling points for RNA-seq.

FIGS. 3a, 3b, and 3c depict performance of engineered strains grown on formate at bioreactor (500 mL) scale. Bioreactor cultivation was conducted using a pH-controlled fed-batch mode with a 35% (w/v) formic acid feed. Strain performance was compared by FIG. 3a) cell growth (OD₆₀₀), FIG. 3b) formic acid feed added (grams), and FIG. 3c) the maximum growth parameters achieved, as indicated. Strain CHC122 is shown as the average of duplicate bioreactors, with standard error indicated as error bars. In all other cases, data is shown as averages of triplicate bioreactors, with standard deviations indicated as error bars. Values with an asterisk indicate a p≤0.20 difference in the value, as compared to the CHC023 (ΔphaCAB) control.

FIGS. 4a, 4b, 4c, and 4d depict performance of engineered strains on alternate carbon sources grown at microplate (900 μL) scale. Microplate growth curves of strains obtained from rational metabolic engineering, cultivated on minimal media supplemented with either FIG. 4a) 42 mM acetate, FIG. 4b) 12 mM benzoate, FIG. 4c) 21 mM succinate, or FIG. 4d) 14 mM fructose using a BioLector II microtiter plate reader. Data is shown as the average of triplicate wells, with standard deviations indicated as error bars.

FIGS. 5a, 5b depict effects of pH during growth on formate at microplate (200 μL) scale. Microplate growth curves of wildtype (H16) and CHC076 (ΔphcA) strains, cultivated on minimal media supplemented with 50 mM sodium formate at an initial pH of either FIG. 5a) 6.8 or FIG. 5b) 5.8 using a Bioscreen C Pro microplate reader. Data is shown as the average of quadruplicate wells, with standard deviations indicated as error bars.

### DETAILED DESCRIPTION

Conversion of CO₂ to value-added products presents an opportunity to reduce GHG emissions while generating revenue. Formate, which can be generated by the electrochemical reduction of CO₂, has been proposed as a promising intermediate compound for microbial upgrading. Here we present progress towards improving the soil bacterium Cupriavidus necator H16, which is capable of growing on formate as its sole source of carbon and energy using the Calvin-Benson-Bassham (CBB) cycle, as a host for formate utilization. Using adaptive laboratory evolution, we generated several isolates that exhibited faster growth rates on formate. The genomes of these isolates were sequenced, and resulting mutations were systematically reintroduced by metabolic engineering, to identify those that improved growth. The metabolic impact of several mutations was investigated further using RNA-seq transcriptomics. We found that deletion of a transcriptional regulator implicated in quorum sensing, PhcA, reduced expression of several operons and led to improved growth on formate. Growth was also improved by deleting large genomic regions present on the extrachromosomal megaplasmid pHG1, particularly two hydrogenase operons and the megaplasmid CBB operon, one of two copies present in the genome. Based on these findings, we generated a rationally engineered ΔphcA and megaplasmid-deficient strain that exhibited a 24% faster maximum growth rate on formate. Moreover, this strain achieved a 7% growth rate improvement on succinate and a 19% increase on fructose, demonstrating the broad utility of microbial genome reduction. This strain has the potential to serve as an improved microbial chassis for biological conversion of formate to value-added products.

Cupriavidus necator (formerly known as Ralstonia eutropha, Alcaligenes eutrophus, Wautersia eutropha, and Hydrogenomonas eutropha) is one of the best-studied native formatotrophs. C. necator is able to grow autotrophically using the Calvin-Benson-Bassham (CBB) cycle to fix CO₂ from its environment when an energy source such as H₂ is also provided. C. necator is also capable of growth on formate as its sole source of carbon and energy, where intracellular formate dehydrogenation is carried out by several native formate dehydrogenases to generate both energy in the form of NADH reducing equivalents and CO₂ for assimilation by the CBB cycle. C. necator is amenable to formate concentrations up to at least 2 g/L, and the effects of formate toxicity can be mitigated in pH-controlled fed-batch cultivations (pH-stat) that maintain a low concentration of formic acid. C. necator is also genetically tractable, has been successfully engineered to produce myriad products, and has long been employed in large-scale and high cell density commercial production of polyhydroxyalkanoate (PHA) biopolymers. Recently, this species has been metabolically engineered to autotrophically produce a variety of chemicals from CO₂ including: methyl ketones, alka(e)nes, terpenes, acetoin, fatty acids, isopropanol, lipochitooligosaccharides, sucrose, polyhydroxyalkanoates, 1,3-butanediol, trehalose, D-mannitol, glucose, and lycopene, as well as isobutanol and 3-methyl-1-butanol from electrochemically generated formate. Additionally, progress has been made towards improving autotrophic growth of C. necator via optimization of its native metabolism, and by introduction of heterologous enzymes or pathways.

As a soil bacterium, C. necator evolved in an environment with variable and transitory sources of carbon and energy. Consequently, it has been suggested that its genome is that of a strong generalist, with a diverse chemolithotrophic metabolism capable of versatile growth on a wide variety of substrates and electron acceptors. As such, we hypothesized that wild-type C. necator H16 is unlikely to be fully optimized for growth on formate as the sole source of carbon and energy. Indeed, recent analysis of protein allocation and utilization during growth on several substrates, including formate, suggested that large fractions of the proteome are underutilized, and that autotrophy may be a recent evolutionary acquisition in H16.

The genetic, physiologic, and molecular mechanisms underlying formatotrophy are not fully understood, making rational metabolic engineering to improve conversion of formate difficult. Adaptive laboratory evolution (ALE) is a powerful tool for generating desirable phenotypic improvements without complete, a priori knowledge of the mechanisms that govern them.

Disclosed herein are methods and compostions to improve C. necator H16 as a host for formate conversion. Methods disclosed herein are applicable to other C. necator sp. To this end, we first subjected it to ALE using serial batch transfers with formate as the sole source of carbon and energy, in order to naturally select for mutations that enabled cells to grow more rapidly. Evolved isolates were analyzed by whole genome sequencing to identify genetic targets for rational metabolic engineering. We then generated a series of rationally engineered strains (Table 1) and found that they recapitulated and ultimately exceeded the growth improvements observed in the evolved strains. RNA-seq transcriptomics were performed on engineered strains to help elucidate the underlying mechanisms that contributed to improved growth on formate. We found deletion of the gene encoding the transcriptional regulator PhcA, the soluble and membrane-bound hydrogenase operons, the megaplasmid copy of the CBB operon, and finally the entire megaplasmid pHG1, were the most effective genetic modifications. Collectively, these results point towards genome minimization as a promising strategy for generating C. necator strains with improved growth under controlled conditions. Surprisingly, we also found that modifications that improved growth on formate also improved growth on succinate and fructose, yielding an improved C. necator platform strain with substantial academic and industrial potential.

TABLE 1
Strains

| Strain | Genotype | Alias |
|---|---|---|
| CHC001 | Cupriavidus necator ATCC 17699 | H16, WT |
| CHC004 | Cupriavidus necator DSM 542 | G + 7 |
| CHC020 | CHC001 ΔH16_A0006 | ΔRE |
| CHC023 | CHC020 ΔphaCAB | ΔphaCAB |
| CHC045 | CHC001, Formate ALE, Generation 400, | HA6 |
| | Population A, Colony #6 | |
| CHC046 | CHC001, Formate ALE, Generation 400, | HB3 |
| | Population B, Colony #3 | |
| CHC048 | CHC001, Formate ALE, Generation 400, | HC8 |
| | Population C, Colony #8 | |
| CHC050 | CHC004, Formate ALE, Generation 400, | GD2 |
| | Population D, Colony #2 | |
| CHC053 | CHC004, Formate ALE, Generation 400, | GE7 |
| | Population E, Colony #7 | |
| CHC055 | CHC004, Formate ALE, Generation 400, | GF4 |
| | Population F, Colony #4 | |
| CHC076 | CHC020 ΔphcA | ΔphcA |
| CHC077 | CHC020 ΔhoxKGZMLOQRTV ΔhypA1B1F1CDEX | ΔMBH |
| | ΔhoxABCJ | |
| CHC078 | CHC020 ΔhoxFUYHWI ΔhypA2B2F2 | ΔSH |
| CHC079 | CHC020 ΔcbbR′ | ΔCBBp |
| | ΔcbbLpSpXpYpEpFpPpTpZpGpKpAp | |
| CHC091 | CHC020 ΔhypD | ΔhypD |
| CHC092 | CHC020 ΔcbbR′ | ΔCBBp ΔMBH |
| | ΔcbbLpSpXpYpEpFpPpTpZpGpKpAp | ΔSH |
| | ΔhoxKGZMLOQRTV ΔhypA1B1F1CDEX | |
| | ΔhoxABCJ ΔPHG023-087 ΔhoxFUYHWI | |
| | ΔhypA2B2F2 | |
| CHC099 | CHC092 ΔphcA | ΔCBBp ΔMBH |
| | | ΔSH ΔphcA |
| CHC105 | CHC020 ΔpHG1 | ΔpHG1 |
| CHC113 | CHC105 ΔphcA | ΔpHG1 ΔphcA |
| CHC122 | CHC076 ΔphaCAB | ΔphcA ΔphaCAB |
| CHC123 | CHC105 ΔphaCAB | ΔpHG1 ΔphaCAB |
| CHC124 | CHC113 ΔphaCAB | ΔphcA ΔpHG1 |
| | | ΔphaCAB |

Materials and Methods

Plasmid Construction.

Plasmid synthesis using the pK18sB vector (GenBank Accession MH166772, Addgene Plasmid #177838) backbone was performed by Twist Biosciences. Conjugative plasmids were built using the compact conjugation vector pK18msB (GenBank Accession #OK423783, Addgene Plasmid #177839). For plasmids built manually, Phusion Polymerase (New England Biolabs) was used for amplifying fragments from C. necator genomic DNA. Plasmids were assembled via the Gibson Method using Gibson Assembly Master Mix (New England Biolabs). Plasmids were transformed into chemically competent NEB 5-alpha FIq E. coli (New England Biolabs) and were selected on LB (Lennox) agar plates supplemented with 50 μg/mL kanamycin (Kan:50). Correct plasmid assemblies were validated by colony PCR, followed by Sanger sequencing (GENEWIZ, Inc.). Detailed construction information for all plasmids is reported in Tables 2, 3, and 4.

TABLE 2
Construction details for plasmids disclosed herein.

| Plasmid | Description | Construction details |
|---|---|---|
| pK18mobsacB | Backbone for C. necator | ATCC 87097. GenBank: FJ437239. |
| | transformation via conjugation | |
| | or electroporation | |
| pK18sB | Backbone for C. necator | GenBank: MH166772. Addgene Plasmid # 177838. |
| | transformation via | |
| | electroporation | |
| pK18msB | Compact backbone for C. necator | GenBank Accession # OK423783. Addgene Plasmid |
| | transformation via | #177839 |
| | conjugation or electroporation | |
| pQP307 | Conjugation knockout plasmid | pQP307 was constructed by amplification of 1021/1047 bp of |
| | (pK18mobsacB) to delete C. necator | H16_A0006 upstream and downstream targeting sequences |
| | H16_0006 (Type I | from the C. necator genome with primer pairs |
| | restriction endonuclease subunit) | oQP1714/oQP1715 and oQP1718/oQP1719. Next, the poly- |
| | and replace with polyattB sites | attB cassette insert was amplified from from plasmid pGW64 |
| | | using primers oQP1716/oQP1717 (Elmore et al., 2020). |
| | | Products were assembled into the EcoRI and HindIII sites of |
| | | pK18mobsacB using NEBuilder HiFi DNA Assembly Master |
| | | Mix (New England Biolabs). The products were transformed |
| | | into E. coli and a positive clone was confirmed by DNA |
| | | sequencing. |
| pCHC004 | Electroporation knockout | Plasmid pCHC004 was constructed by first amplifying 750 bp |
| | plasmid (pK18sB) to delete the | of phaCAB upstream and downstream targeting sequences |
| | C. necator phaCAB operon, to | from purified C. necator genomic DNA with primer pairs |
| | prevent accumulation of PHB | oCHC021/oCHC022 and oCHC023/oCHC024, each |
| | | containing 30 bp overlapping sequences for Gibson Assembly. |
| | | Primers were designed to introduce a PmeI site between |
| | | targeting regions. Products were purified by gel extraction |
| | | and assembled into the EcoRI and HindIII sites of pK18sB |
| | | using the Gibson Method. The products were transformed into |
| | | E. coli and a positive clone was confirmed by DNA |
| | | sequencing. |
| pCHC005 | Conjugation knockout plasmid | The polyattB site was removed from plasmid pQP307 |
| | (pK18mobsacB) to delete | (ΔH16_A0006::polyattB) by Q5 PCR mutagenesis, using |
| | H16_0006 (Type I restriction | primers oCHC027-028 with overlaps designed to introduce a |
| | endonuclease subunit) | PmeI site between targeting regions. |
| pCHC022 | Electroporation knockout | Plasmid pCHC022 was designed by incorporating 750 bp of |
| | plasmid (pK18sB) to delete the | phcA upstream and downstream targeting sequences from the |
| | C. necator LysR-type | C. necator genome, and inserting them at the EcoRI and |
| | transcriptional regulator PhcA | HindIII sites of the pk18sB vector backbone. Plasmid |
| | | construction and sequencing was completed by Twist |
| | | Biosciences. |
| pCHC023 | Electroporation knockout | Plasmid pCHC023 was designed by incorporating 750 bp of |
| | plasmid (pK18sB) to delete the | MBH upstream and downstream targeting sequences from the |
| | C. necator membrane-bound | C. necator genome, and inserting them at the EcoRI and |
| | hydrogenase operon (ΔMBH) | HindIII sites of the pk18sB vector backbone. Plasmid |
| | | construction and sequencing was completed by Twist |
| | | Biosciences. |
| pCHC024 | Electroporation knockout | Plasmid pCHC024 was designed by incorporating 750 bp of |
| | plasmid (pK18sB) to delete the | SH upstream and downstream targeting sequences from the |
| | C. necator soluble hydrogenase | C. necator genome, and inserting them at the EcoRI and |
| | operon (ΔSH) | HindIII sites of the pk18sB vector backbone. Plasmid |
| | | construction and sequencing was completed by Twist |
| | | Biosciences. |
| pCHC025 | Electroporation knockout | Plasmid pCHC025 was designed by incorporating 750 bp of |
| | plasmid (pK18sB) to delete the | CBBp upstream and downstream targeting sequences from |
| | C. necator megaplasmid copy of | the C. necator genome, and inserting them at the EcoRI and |
| | the CBB operon (ΔCBBp) | HindIII sites of the pk18sB vector backbone. |
| pCHC027 | Conjugation knockout plasmid | Plasmid pCHC027 was constructed by first amplifying 750 bp |
| | (pK18msB) to delete the C. necator | of pemK upstream and downstream targeting sequences from |
| | addiction system toxin | purified C. necator genomic DNA with primer pairs |
| | PemK, to enable deletion of the | oCHC154/155 and oCHC156/157, each containing 30 bp |
| | pHG1 megaplasmid | overlapping sequences for Gibson Assembly. Products were |
| | | purified by gel extraction, and assembled into the EcoRI and |
| | | HindIII sites of pK18msB using the Gibson Method. The |
| | | products were transformed into E. coli and a positive clone |
| | | was confirmed by DNA sequencing. |
| pCHC030 | Electroporation knockout | Plasmid pCHC030 was designed by incorporating 750 bp of |
| | plasmid (pK18sB) to delete the | CBBp upstream and SH downstream targeting sequences |
| | CBBp, membrane-bound | from the C. necator genome, and inserting them at the EcoRI |
| | hydrogenase, soluble | and HindIII sites of the pk18sB vector backbone. |
| | hydrogenase, and intervening | |
| | sequences (ΔCBBpΔMBHΔSH) | |
| pCHC036 | Conjugation knockout plasmid | Plasmid pCHC036 was constructed by first amplifying 750 bp |
| | (pK18msB) to delete the C. necator | of helD upstream and parAB downstream targeting sequences |
| | megaplasmid replication | from purified C. necator genomic DNA with primer pairs |
| | region containing helD, repA, | oCHC188/189 and oCHC190/191, each containing 30 bp |
| | repB, and parAB in order to | overlapping sequences for Gibson Assembly. Products were |
| | promote loss of the entire pHG1 | purified by gel extraction, and assembled into the EcoRI and |
| | megaplasmid | HindIII sites of pK18msB using the Gibson Method. The |
| | | products were transformed into E. coli and a positive clone |
| | | was confirmed by DNA sequencing. |
| pCHC039 | Conjugation knockout plasmid | Plasmid pCHC039 was constructed using pK18msB digested |
| | (pK18msB) to delete the C. necator | with EcoRI/HindIII followed by dephosphorylation with CIP. |
| | LysR-type transcriptional regulator PhcA | The phcA upstream/downstream genomic targeting regions |
| | | were released from pCHC022 by digestion with |
| | | EcoRI/HindIII, followed by gel extraction. The products were |
| | | assembled together using T4 ligase and transformed into |
| | | E. coli. A positive clone was confirmed by DNA sequencing. |
| pCHC042 | Conjugation knockout plasmid | Plasmid pCHC042 was constructed by first amplifying 750 bp |
| | (pK18msB) to delete the C. necator | of phaCAB upstream and downstream targeting sequences |
| | phaCAB operon, to | from purified C. necator genomic DNA with primer pairs |
| | prevent accumulation of PHB | oCHC021/oCHC022 and oCHC023/oCHC024, each |
| | | containing 30 bp overlapping sequences for Gibson Assembly. |
| | | Primers were designed to introduce a PmeI site between |
| | | targeting regions. Products were purified by gel extraction |
| | | and assembled into the EcoRI and HindIII sites of pK18msB |
| | | using the Gibson Method. The products were transformed into |
| | | E. coli and a positive clone was confirmed by DNA |
| | | sequencing. |

TABLE 3
Oligonucleotide primers disclosed herein.

| Primer | SEQ ID NO. | Sequence (5′-3′) |
|---|---|---|
| oCHC021: | SEQ ID NO: 1 | AGGAAACAGCTATGACATGATTACGAATTCCGCCGGTCGCTT |
| phaCAB Up F + | | CTACTC |
| EcoRI | | |
| oCHC022: | SEQ ID NO: 2 | CTGGTTGAACCAGGCCGGCAGGGTTTAAACGATTTGATTGTC |
| phaCAB Up R + | | TCTCTGCCGTCA |
| Link | | |
| oCHC023: | SEQ ID NO: 3 | ACGGCAGAGAGACAATCAAATCGTTTAAACCCTGCCGGCCT |
| phaCAB Dn F + | | GGTT |
| Link | | |
| oCHC024: | SEQ ID NO: 4 | CGTTGTAAAACGACGGCCAGTGCCAAGCTTGCCTGGATGTTC |
| phaCAB Dn R + | | TTTTCCAGG |
| HindIII | | |
| oCHC027: RE | SEQ ID NO: 5 | GTTTAAACTGCCTTCGCCGGTGAAATTGCCAAG |
| ko F + PmeI | | |
| oCHC028: RE | SEQ ID NO: 6 | TCAGGCGCTCCCTGCTTGTTTGG |
| ko R | | |
| oCHC031: RE | SEQ ID NO: 7 | GGTGCAGAGCCCTACCTGAGTCC |
| Up Geno F | | |
| oCHC032: RE | SEQ ID NO: 8 | CGACCTCGTCGTAGCGCAGC |
| Down Geno R | | |
| oCHC142: | SEQ ID NO: 9 | GATTGCGTCGCCGTCCACCAGGAAATG |
| phcA Up Geno | | |
| F | | |
| oCHC143: | SEQ ID NO: 10 | GAGGTGGAATCGTAGGCTGAGCAGGCG |
| phcA Dn Geno | | |
| R | | |
| oCHC144: | SEQ ID NO: 11 | GCTGCCATGAGCGAAGTCACGTTGATCG |
| pemK Up Geno | | |
| F | | |
| oCHC145: | SEQ ID NO: 12 | GCACACTTGGTTCCTGACAGGCCGAAAC |
| pemK Dn Geno | | |
| R | | |
| oCHC147: | SEQ ID NO: 13 | GACCTCCATTGACGCCCATAATGCGCTC |
| parAB Dn Geno | | |
| R | | |
| oCHC148: SH | SEQ ID NO: 14 | GTAGATCACCGCCTTGTTGTACCACGCG |
| Up Geno F | | |
| oCHC149: SH | SEQ ID NO: 15 | TAGGCATGCGCATGGGTACGAGGAGTC |
| Dn Geno R | | |
| oCHC150: | SEQ ID NO: 16 | CATCAGCCTGTTATCACTGCACACGCTGTC |
| MBH Up Geno | | |
| F | | |
| oCHC151: | SEQ ID NO: 17 | AGAAGGTCAAAGTCTTCCTCAACGTAGATGCCG |
| MBH Dn Geno | | |
| R | | |
| oCHC152: | SEQ ID NO: 18 | ACAAGATCTATGCCTGAATCCGAAGACCTGGG |
| CBBp Up Geno | | |
| F | | |
| oCHC153: | SEQ ID NO: 19 | CTGCGCACTGAAACCCAGCAACTTCATG |
| CBBp Dn Geno | | |
| R | | |
| oCHC154: | SEQ ID NO: 20 | AGGAAACAGCTATGACATGATTACGAATTCAGCTGCTACCTC |
| pemK Up F + | | GAGGCTGCACAAGAG |
| EcoRI | | |
| oCHC155: | SEQ ID NO: 21 | GACAACGCGCCATGCGGTCAAGCATGGAGGTTACATGGCCT |
| pemK Up R + | | CCGCGCCGACACG |
| Link | | |
| oCHC156: | SEQ ID NO: 22 | ACGCAGCGTGTCGGCGCGGAGGCCATGTAACCTCCATGCTT |
| pemK Dn F + | | GACCGCATGGCGC |
| Link | | |
| oCHC157: | SEQ ID NO: 23 | CGACGGCCAGTGCCAAGCTTTATTTGCATAGTGTTTGCCGAC |
| pemK Dn R + | | TACTGTTTGTACATCGAC |
| HindIII | | |
| oCHC163: | SEQ ID NO: 24 | CCTGAACATGTTCTGGCACCGCAGC |
| phcA Check F | | |
| oCHC164: | SEQ ID NO: 25 | GCGGATCGTCAAAGATTTCACGCAGCC |
| phcA Check R | | |
| oCHC165: | SEQ ID NO: 26 | CAGCTATGGCATTGTCGAGAGACATGGCG |
| MBH Check F | | |
| oCHC166: | SEQ ID NO: 27 | GCGATCTGCGGCAGAAAGGAAGGTCC |
| MBH Check R | | |
| oCHC167: SH | SEQ ID NO: 28 | GCTGCTTCCTCAACCACATCCTCGCC |
| Check F | | |
| oCHC168: SH | SEQ ID NO: 29 | GAATGTCCAGCGGGGACAGCTTCAACC |
| Check R | | |
| oCHC170: | SEQ ID NO: 30 | CGCATCCGCACGTGCTAGTGGCTTC |
| CBBp Check R | | |
| oCHC171: | SEQ ID NO: 31 | CAAGCTGCTGGAGGCTTCGCTACTTCG |
| parAB Check F | | |
| oCHC173: | SEQ ID NO: 32 | CTAGTATTGTGATTGGCTTGCCGATGACTACGG |
| pemK Check F | | |
| oCHC174: | SEQ ID NO: 33 | CTTTCCCTTCGGTCCCTGAAGCTTGATCG |
| pemK Check R | | |
| oCHC179: | SEQ ID NO: 34 | GAGGAGATCCTGCGCGGCATCAAGAC |
| CBBp Check F2 | | |
| oCHC188: | SEQ ID NO: 35 | AGGAAACAGCTATGACATGATTACGAATTCCGCCGTCGTCG |
| pHG1 Rep Up | | CGAACTCGGTC |
| F + EcoRI | | |
| oCHC189: | SEQ ID NO: 36 | GCGAGCGTGCAATCGGATCGGCGCCAACGCGGCGGATCGCA |
| pHG1 Rep Up | | GTGTGGCAGTAAGTG |
| R + Link | | |
| oCHC190: | SEQ ID NO: 37 | GTGCCACTTACTGCCACACTGCGATCCGCCGCGTTGGCGCCG |
| pHG1 Rep Dn | | ATCCGATTGC |
| F + Link | | |
| oCHC191: | SEQ ID NO: 38 | CGTTGTAAAACGACGGCCAGTGCCAAGCTTTTAGCGGCAGA |
| pHG1 Rep Dn | | GTCCGGCGCTAAAC |
| R + HindIII | | |
| oCHC224: | SEQ ID NO: 39 | CGATGCCTTCCTGGCCCAGGCAC |
| phaCAB Geno | | |
| F | | |
| oCHC225: | SEQ ID NO: 40 | CTGGCGGGACCATTCCAGCCATGTG |
| phaCAB Check | | |
| R | | |
| oCHC226: | SEQ ID NO: 41 | GATCCGCCAGGACGTGCTCGACAAG |
| phaCAB Check | | |
| F | | |
| oCHC227: | SEQ ID NO: 42 | GCTCATCATGCCCTGCATCATCGGGC |
| phaCAB Geno | | |
| R | | |
| oQP1714 | SEQ ID NO: 43 | AGGAAACAGCTATGACATGATTACGAATTCGACGATGACGA |
| | | AGATTTCTCCGAG |
| OQP1715 | SEQ ID NO: 44 | CTGCCACTATCGTCGTCAGGCGCTCCCTGCTTG |
| oQP1716 | SEQ ID NO: 45 | GCAGGGAGCGCCTGACGACGATAGTGGCAGCATGC |
| oQP1717 | SEQ ID NO: 46 | TCACCGGCGAAGGCAGGATTTCATGTAGTTGTAGGCGTCTTC |
| oQP1718 | SEQ ID NO: 47 | AACTACATGAAATCCTGCCTTCGCCGGTGAAATTG |
| oQP1719 | SEQ ID NO: 48 | CGTTGTAAAACGACGGCCAGTGCCAAGCTTCAACGGTATCG |
| | | ATCTTGACTACGAAGC |

TABLE 4
Targeting sequences for knockout plasmids disclosed herein.

| Plasmid | SEQ ID NO. | Plasmid Targeting Sequences (Upstream, PmeI Site, Downstream) |
|---|---|---|
| pCHC004 | SEQ ID NO: 49 | CGCCGGTCGCTTCTACTCCTATCGGCGCGATGGCGTGACCGGCCGCAT |
| (ΔphaCAB) | Upstream | GGCCAGCCTGGTCTGGCTGGCGGACTGAGCCCGCCGCTGCCTCACTCG |
| and | | TCCTTGCCCCTGGCCGCCTGCGCGCGCTCGGCTTCAGCCTTGCGTCGG |
| pCHC042 | | CGGCGGCCGGGCGTGCCCATGATGTAGAGCACCAGCGCCACCGGCGC |
| (ΔphaCAB) | | CATGCCATACATCAGGAAGGTGGCAACGCCTGCCACCACGTTGTGCT |
| | | CGGTGATCGCCATCATCAGCGCCACGTAGAGCCAGCCAATGGCCACG |
| | | ATGTACATCAAAAATTCATCCTTCTCGCCTATGCTCTGGGGCCTCGGC |
| | | AGATGCGAGCGCTGCATACCGTCCGGTAGGTCGGGAAGCGTGCAGTG |
| | | CCGAGGCGGATTCCCGCATTGACAGCGCGTGCGTTGCAAGGCAACAA |
| | | TGGACTCAAATGTCTCGGAATCGCTGACGATTCCCAGGTTTCTCCGGC |
| | | AAGCATAGCGCATGGCGTCTCCATGCGAGAATGTCGCGCTTGCCGGA |
| | | TAAAAGGGGAGCCGCTATCGGAATGGACGCAAGCCACGGCCGCAGC |
| | | AGGTGCGGTCGAGGGCTTCCAGCCAGTTCCAGGGCAGATGTGCCGGC |
| | | AGACCCTCCCGCTTTGGGGGAGGCGCAAGCCGGGTCCATTCGGATAG |
| | | CATCTCCCCATGCAAAGTGCCGGCCAGGGCAATGCCCGGAGCCGGTT |
| | | CGAATAGTGACGGCAGAGAGACAATCAAATC |
| | PmeI site | GTTTAAAC |
| | SEQ ID NO: 50 | CCTGCCGGCCTGGTTCAACCAGTCGGCAGCCGGCGCTGGCGCCCGCG |
| | Downstream | TATTGCGGTGCAGCCAGCGCGGCGCACAAGGCGGCGGGCGTTTCGTT |
| | | TCGCCGCCCGTTTCGCGGGCCGTCAAGGCCCGCGAATCGTTTCTGCCC |
| | | GCGCGGCATTCCTCGCTTTTTGCGCCAATTCACCGGGTTTTCCTTAAGC |
| | | CCCGTCGCTTTTCTTAGTGCCTTGTTGGGCATAGAATCAGGGCAGCGG |
| | | CGCAGCCAGCACCATGTTCGTGCAGCGCGGCCCTCGCGGGGGCGAGG |
| | | CTGCAGGCCGCCACGCGCAGCCATGCGCGAACGGGCCACCAGATGGC |
| | | CGGCACGACAACAAGCAGATGGCGCGGGCGATACCGATTTGCGCACT |
| | | GCACCCCATGCGGTGCAGCAGCGCGCAAACAGCGATGACACAAGGAC |
| | | AGAGCACCGATGGCCACGACCAAAAAAGGCGCAGAGCGACTGATCA |
| | | AAAAGTATCCGAACCGTAGGCTCTACGACACCCAGACCAGCACCTAC |
| | | ATCACCCTGGCCGACGTCAAGCAGCTGGTCATGGATTCAGAAGAATT |
| | | CAAGGTCGTCGACGCCAAGTCTGGTGACGAACTGACCCGCAGCATCT |
| | | TGCTGCAGATCATCCTGGAAGAAGAAACGGGCGGCGTGCCGATGTTC |
| | | TCCAGCGCGATGCTGTCGCAGATCATCCGCTTCTACGGCCATGCCATG |
| | | CAGGGCATGATGGGCACCTACCTGGAAAAGAACATCCAGGC |
| pCHC005 | SEQ ID NO: 51 | GACGATGACGAAGATTTCTCCGAGCAGTGTTTTTCGTCCGGCATACTT |
| (ΔRE) | Upstream | TTTTCATGTCGCCAAGCAAGCTGAGCGATTCCTGAAGGCTCAAACCAA |
| | | TGGCACTGGCATTCCACACGTTGACCGAGAGCTTCTCGAGGGGATAA |
| | | AGGTCTTTTGTCCTGGCTCTACGGAGCAGCAATTACTTGCGGAAATCC |
| | | TCGACACTCTCGACACCGCCATCTACGAAACTGAAGCGATCATCGCC |
| | | AAGCTCAAGGCGGTCAAGCAAGGCCTGCTGCATGACCTCTTGACGCG |
| | | CGGCATCGACGCCAACGGCGAATTGCGCCCACCTCAGGCCGAGGCAC |
| | | CGCATCTCTACGAGTCGTCACCGTTGGGTTGGATTCCGAATGAGTGGG |
| | | GTCTTGCTCCTACAGCAACTCGCTGCCATCTGATAACCAAAGGCACTA |
| | | CCCCTGCGGCTAATGAGATGTGGCAGGGTGGCGCGGGAATTAGGTTT |
| | | CTGCGAGTCGATAATCTTTCTTTCGATGGACAACTGGATCTAGATGCA |
| | | AGCACGTTTCGAGTTAGCCTTGCCACGCACAAAGGTTTTCTGGCTCGT |
| | | TCAAGATGCCTTGAAGGTGATGTGCTGACGAACATCGTTGGCCCACCT |
| | | CTAGGGAAACTGGGGCTTGTTACCAAAGAAATTGGTGAGGTCAATAT |
| | | TAATCAAGCAATTGCGTTATTTCGACCAACCGAACAACTACTGCCAAA |
| | | GTTCCTATTAATCTGGCTTAGTAGCTCAATCTCGCAGTCTTGGCTGAG |
| | | GAACCGAGCCAAGCAGACGTCGGGACAAGTGAATCTGACCCTCGCTC |
| | | TATGCCAGGAGCTTCCTCTACCTCGGATGACGATCAATGAGCAACAG |
| | | GCAATCGTTGACCGAGTTGATGCCGCGCAGGAACAAATCTGGTGTGA |
| | | GGAGGAACTGATCCGAAAGATGCGACTTGAGAAATCTGGCCTTATGG |
| | | ATGACCTCCTCACCGGCCGCGTCCGCGTCAAGCCGCAGCTGGCGGAA |
| | | ACCAAACAAGCAGGGAGCGCCTGA |
| | PmeI site | GTTTAAAC |
| | SEQ ID NO: 52 | TGCCTTCGCCGGTGAAATTGCCAAGCCTTCAGATCGGTGACCTCCGGT |
| | Downstream | TCACGCTCCAGCGGAGCGCGCGCCGCAGAACTATGCAGATCACCGTG |
| | | GAGCGCAGTGGCGACTTGATGCTCTGCGCACCGCCGGAGGTGGACGA |
| | | GGCCGCGCTGCGAGCATTCGTGCTGGAGAAGCGCTTCTGGATCTACA |
| | | CCAAGCTGGCCGAGAAGGACCGCTTGCAGCGCCAGGTTCCGCGCAAG |
| | | GAATTCGTCGGAGGCGAGGGATTCTTGTATCTCGGCCGCAGCCATCG |
| | | GCTGAAGGTGGTCGATGAACAGAATGTGCCACTGAAGTTGAATGGAG |
| | | GCCGCTTTTGTCTGCGCCGTGACGCCCTACCCGCCGCGCGCGAGCATT |
| | | TCATCCGCTGGTACGGCGAGCGTGCCAAGGCCTGGCTTTCGGGGCGT |
| | | GTAGCTGACTACCAGTCGCGAATGGAGGTGACGCCTGCCGGCGTCAA |
| | | GGTGCAGGACCTTGGATATCGCTGGGGTTCGTGTGGCAAGGGCGACT |
| | | GGCTGTACTTCCACTGGAAGGCAATCCTGCTGCCGGCGCGCATCGCTG |
| | | AGTATGTCGTGGTGCATGAGATTGCCCATCTGCATGAGCCGCACCACA |
| | | CGCCTGCGTTCTGGCTTCGAGTGGAGCGTGCCATGCCGGACTATGCGC |
| | | AACGCAAGGCCTGGCTGGCCGAGCATGGAATCGATGTTGAAGGAATC |
| | | TAAAGAACGATGGCTGACTATTTCACCAGTGACTACTTCAAGCTGCTG |
| | | AACAAGTGGAAGGGGCAGAAGCGTGACGAGTCCAACCCCGAGCAGA |
| | | ACCGCGCTTATGAAGATCTGAAGAAGGCCTACGAGGTGACGGAGGCG |
| | | TGGGCGGACAAGGTTAAGGCCGAGTTGTTCCCTGTCGGGCGCGTCGA |
| | | GATTCGTAAGCGCCCGACCAACCAGGGCAACAACTTTGCCAGCTACA |
| | | ACTGGGCCAAAATCTACCCTTCATCTGAGGCGCCGAAAGAGTTGGCTT |
| | | ACACAGTTGGCATCGGCGCCGATGACGGCTTCGTAGTCAAGATCGAT |
| | | ACCGTTG |
| pCHC022 | SEQ ID NO: 53 | ACGACTTCGCCAAGGAACAGGTCGTAGGTCTGCTGCGTGGCCGGCTC |
| (ΔphcA) | Upstream | CGGCAGCAGCCGGCATTCCAGCCAGGCCGCGCAGCCCTCCAGCAGCG |
| | | GCGCGCCCACCGCCGTGCCGGCAAAGGTGCCAAGGCCGTAGGCGTCG |
| | | AACTTGTCGGTGCCTTCCTGCTCCATCAGCGCCAGGCCCGAGCTGGAG |
| | | CCCAGTGCCTCGGTCAGGTCGACCTGGCTGACGGTGGGGACCTGCAA |
| | | CACGAACTCGCCGCTGTCTTCCAGCAGGTGCCGGGTCCAGGTGCTCTT |
| | | GTCCAGCACCACTGCCACCTTGGGCGGGGCGAAGTCGAGCGGCATGG |
| | | CCCAGGCGGCGGCCATGATATTGCGCTTGCCGCCGGCGGCGGCGCTG |
| | | ACCAGCACAGTGGGGCCGTGGTTCAGCAAGCGGTAGGCTTTCGGGAG |
| | | TGATACGGGCAGGCGGAAATGTTCAGGCATGATGGCCGGGATGAGCC |
| | | GTCAGAAAAGAATGATAAAAATGGGAACGGCGGACCCACTATACCCG |
| | | GATGTACGAGTGCATGTTGCGGCGCGGGAAATGTTCACATATGCGGT |
| | | CAATTGTGGAAAAAGAGCGCAATTTTTCAGAAATATGGCGTAGACGG |
| | | CCATTTCAGAAATGCCGAATTTGCTTTCCGAGCTTGTTTTTTCCTCTTA |
| | | CACTATTAAGACGCCGTTGAAATCTGATGTGCAGCCAGTGCAAGTGG |
| | | TGGGGCCATCTAGCTAAGAATAATCTGACCGAGGCCTGATC |
| | SEQ ID NO: 54 | GCAGCATCCTGCGGCGAGCAGCCCAAACAAAAAACCGGCGCCTGGCG |
| | Downstream | CCGGTTTTTTGTTGCCCGTCTGCGCTCCGCGGTGGAGCGTGCAGGCTT |
| | | ATCGTTTGGGTCTGTGGGGACAGTCTGTTTTGGTGCAATTGCCGTACA |
| | | GCGACAGTGCATGTTCCTGCAGCGTAAAGCCGCGCTCGCGCGCGATG |
| | | CTTTGCTGGCGGTGCTCGATTTCAGAGTCGAAGAACTCCTCGACGCGG |
| | | CCGCAGTCGAGGCACACCAGGTGGTCATGGTGCTTGCCTTCGTTGAGT |
| | | TCGAAAATCGCCTTGCCGGATTCAAAGTTGTTGCGCGAGAGCAGGCC |
| | | CGCCTGCTCGAACTGGGTCAGCACGCGGTAGACGGTGGCCAGGCCGA |
| | | TGTCCATATGCTCGTTCAGCAGGATACGGTAGACGTCTTCCGCGCTCA |
| | | GGTGCCGCTGCTCACTGGTCTGAAAAATTTCAAGAATCTTCAGCCTGG |
| | | GCACGGTCGCCTTCAGGCCGATGTTCTTGAGGTCCGCCGGACTCGGCA |
| | | TGTGGGTGACTCCCTAGAGTACAATGACTGGATAGTTGAATCATAAG |
| | | GGTTTTGGCAGCAAAAGTCGCTCGCGGTAGTGATGTCCCAGGTCGAC |
| | | GTCACGCGCCCGGCACGGTTGCCGTCCCATGTGGTTGTCGCGGCAGGC |
| | | GCAACAAGGCGTGTGTGGTGCAATCGGCGCATATTGCGCCGTTTTTGT |
| | | GCCCGCTTTGCGGTACCGTGCCGCGGTACTTTTCTT |
| pCHC023 | SEQ ID NO: 55 | GAGCTATGTCGCACCTTCCTGCTGGAACACACGGCGGAATATTTGGA |
| (ΔMBH) | Upstream | ACGGGAATACGGCGGGCTGCTGCCCGGCGGGCTTGTGGCCTAGGGGA |
| | | TTCGGCAAGTCGGGGATCCTGGTAGATGGCGTCGGCCTGCGCATGTGT |
| | | CATGGCGCCGGTGGCGAGATAAGCATCCGGCACCACGGCAGTTGGGC |
| | | TTGCGGGCTGGTCTGCAGTAGCTGCGTGGCATTGCTCGGACGACAGA |
| | | CCCGCTCCTGCCGCGCATGCAGCAGGGGTACAGGAAGGCTGCGAGCA |
| | | GCGCCGGCTCATTGCCTTTCCGTTGGGCGGGGCGAGACGCCGGGGGC |
| | | GGGGGCTCATCCGAGTTCAACGCCGATCACTGAACTTCCTTCTGATGC |
| | | ATTCAAGCGAAAACCCAGTGAGCATCTGGCGTCGGCTAGCGCCAGGC |
| | | GACGGTCCACTTCATGACGGATGAAATATTGTCAAATCAGGATCCGG |
| | | TGTCCTGCGTTGTAGGTTCGCCGAATAGGGCGCTGTCGGGCGGACGC |
| | | ACGAACCTGCGTCACAGATGCTCATACATGCCTTCTCGGTATCAATCT |
| | | TTTTCTAAACAAGCCATCCAACTCAGGATGGTAGCGGGGGTTTTCCCC |
| | | AGGTCTTCGGATTCAGGCATAGATCTTGTTTCAACTATGTCGCCAAGC |
| | | CAGCATTCGTGCGCGAGGGCGGTATCGCTCCCCGGTTGGCGCATCGC |
| | | GACGAATGCCAATACCAATACAGAAATTAGGAGACAGGTT |
| | SEQ ID NO: 56 | TCGGTTGCCGGGGCCCGGCTCCGCCCGTGTTCCGGGGAACGCCTGTTC |
| | Downstream | GAAATTGGCGGAGGCAGGAGGCTGATGGCCTGATTTCCCTGCTGCAC |
| | | CAGGCTAGAAAGCGCTGCTCCGGCTATTTAGACTCCCATGGAACATG |
| | | GTATTGCCATCTGGATATGGGCATGTCACCAATGCGATGATCATGCAA |
| | | ACCTGCTTTGCAGTCCTCACGTACGGACTTGCGCAGCAGATACCGCTA |
| | | TTTCGGGAATAGCATAAGCGAACCAAGACCTGAGAGTGAGCTTCTGC |
| | | CGCATTCGCCAGGAGTTGGCTCGCAGGCGCGGAAATTGCGTTACGGT |
| | | GCAGTCGAGCCTTACTGGCAAAAGCCGCGGATGACAGCGGCGTCGGA |
| | | ACCGAGACAGGAGACTTCCAGCATGTTCCAATTGCTCGCTGGCGTAC |
| | | GCATGAATTCTACTGGCCGCCCGCGGGCCAAGATCATCTTGCTCTACG |
| | | CGCTGCTGATTGCATTCAATATCGGCGCCTGGCTCTGCGCGCTCGCCG |
| | | CGTTTCGCGATCATCCGGTGCTGCTCGGCACCGCACTGCTGGCCTACG |
| | | GCCTTGGGTTGCGCCACGCGGTAGACGCAGATCATCTCGCGGCAATC |
| | | GACAATGTCACCCGCAAGTTGATGCAGGACGGCAGGCGGCCCATCAC |
| | | AGCTGGGCTTTGGTTCTCGCTTGGCCATTCAAGTGTGGTAGTGCTTGC |
| | | TTCGGTGCTGATCGCTGTCATGGCGACCACGCTCCAGG |
| pCHC024 | SEQ ID NO: 57 | GGCACTTGGGGCAATGCCGGTTGCGGCAGGTAATGGAGAGCAACCCA |
| (ΔSH) | Upstream | TTACCTGCCGGCATCGCGCCTGCCCGAAGTGCCAGTCGCTCGCCCGCG |
| | | CGCAATGGCTCGAACACCGGCAGGCTGAGCTGCTGCCCGAGGTCGAG |
| | | TATTTCCATGTGGTCTTCACGGTGCCCGACCCCATCGCGGCGCTCGCC |
| | | TATCAAAACAAGAATCTCTATGACATCCTGTTCCGCACCAGCGCCGAA |
| | | ACCCTGCGCACGATCGCCGCCGATCCGAAACACCTGGGCGCCGAGAT |
| | | CGGCGGCCAGACCTCATCGGGTCCTGCTCATAGGTTCGTAGCCGCGAT |
| | | CGCCAACCAAAAAAACCCTCTCCTGCGGGAAATCCGCACGCTACGTT |
| | | CTGTGGGAACCGGAGGCGGGTGACTGCCTCCGGTCACCCGGTGCTCG |
| | | GGGTGCGATTCCCCGGGTCTACTTACCAAATCGGCCGCGCACCCAATG |
| | | AGAGGCGCTGGCACAAGCTTGCACAGACTTGCCCGCCAAGCGGAAGC |
| | | AGCCTTGCCACATCGGCCGACCCAATGGCAATGCCGCTGCCACCCGC |
| | | CGGATGGCCGTTCTGGAAACGGCTTGAGCGACGTCAAGAATTTCCTTT |
| | | CTCGACAAGCACTTAGCCGGGCCTCCTGGTGGTTTCCCTTAGGCCCTG |
| | | CGAAATTGGCGCACATCCTGCGTTCCACCTGCGCATCGAAGTGACGC |
| | | ACCAAGCAAGGGGCGAACATTAGTAAGGAGGAGACAAC |
| | SEQ ID NO: 58 | CGAGAGGGTAGAACATGTGCCTGGCCATACCTGCACGCATCGCGAAA |
| | Downstream | AAATTTGACAACGACATGGCCCTCATCGACCTGGGCGGCGTGGGGAG |
| | | TGGCCAAAAACGGGGGGCAAATCCGTCAGGAAAGGGGTCTATTGTGT |
| | | ACTGAGACTACCGGAGACCGCCATGCGCATCTCGATCCAAGCCTGTA |
| | | TTGAGCGGGCGGGCGAACAGCCCTCTAAGGTGATTGAAGTTGCGGTG |
| | | ATCGAGCGCAATGCCGATGTCGCTCCGGCCTCAGGACTGGGCCTGTTC |
| | | ATTCGCGAGTCACAAGAGATCCTGCGACAGCTTCAGACTGTGGTCTTG |
| | | ACCGAGCAGGTGGACCAGTTCATCCGGATTACCGGTCGCTGTCAACT |
| | | GTGCGGAGGCAGGCTTGTCATCAAGGACACAAAATCCTTGGTCTATC |
| | | GCACCGCTTTTGGCAAGGCGAGGCTGCGAAGCCCGCGCTTTTACTCTT |
| | | ACTGCAGCGCATGCGGTTACTGCTCAAGTAACAAGGGCACGCTTTCCC |
| | | CGCTGGCACAGGCGTTACCAGAACGCGTACATCCCCAGTGGACCTGG |
| | | CTGCAGTGCCGATATGCAAGCGTGATGTCTTATCGTTTGGCACAGATC |
| | | TTTCTACGCGACGCGTTTGCCGGCGGACGGGAACTCCCATGCTCGAGC |
| | | GTTAAGTTGAATGTAGGCCGGGTCGGGCAGCGGCTGGAGCAAGAGGC |
| | | GCAACGTGCAACGATGGTGATGTCGGCTGTGACCGCGCC |
| pCHC025 | SEQ ID NO: 59 | AGTTGGATGGCTTGTTTAGAAAAAGATTGATACCGAGAAGGCATGTA |
| (ΔCBBp) | Upstream | TGAGCATCTGTGACGCAGGTTCGTGCGTCCGCCCGACAGCGCCCTATT |
| | | CGGCGAACCTACAACGCAGGACACCGGATCCTGATTTGACAATATTT |
| | | CATCCGTCATGAAGTGGACCGTCGCCTGGCGCTAGCCGACGCCAGAT |
| | | GCTCACTGGGTTTTCGCTTGAATGCATCAGAAGGAAGTTCAGTGATCG |
| | | GCGTTGAACTCGGATGAGCCCCCGCCCCCGGCGTCTCGCCCCGCCCAA |
| | | CGGAAAGGCAATGAGCCGGCGCTGCTCGCAGCCTTCCTGTACCCCTG |
| | | CTGCATGCGCGGCAGGAGCGGGTCTGTCGTCCGAGCAATGCCACGCA |
| | | GCTACTGCAGACCAGCCCGCAAGCCCAACTGCCGTGGTGCCGGATGC |
| | | TTATCTCGCCACCGGCGCCATGACACATGCGCAGGCCGACGCCATCTA |
| | | CCAGGATCCCCGACTTGCCGAATCCCCTAGGCCACAAGCCCGCCGGG |
| | | CAGCAGCCCGCCGTATTCCCGTTCCAAATATTCCGCCGTGTGTTCCAG |
| | | CAGGAAGGTGCGACATAGCTCGCTGGCGGGCGACAGCCGCTTGCTAG |
| | | CCATGTGCACGACATGCCAGACACGCTCAATTGGCGTGCCTGCCGCAT |
| | | CGAGCAGCGCGATCTCCCGGTGTGTGCAATTCCAGCGACAGCGTGTG |
| | | CAGTGATAACAGGCTGATGCCCATGCCGGCCATCACCGC |
| | SEQ ID NO: 60 | GCGAGACGTAGTCAGCGAACATGCCATCCGGCCCCTTGCTCATGCTG |
| | Downstream | GAATCACCGAGAGTGTGGTCCGCAGCTGGGGTGCGCTCATGGCAGGT |
| | | GCCTTGTCGGCATCTGTCACCGGTAGCGTGCCCGGCCGTGCAACGCAC |
| | | TGGCGGAGTAGCATGGACAGCTTGGCTTGCAGCATTTCGGCGGTGCC |
| | | GCATAGCGAGGAGGCAAGGGGCGGTGGCGTGGTACATGGGATCGGCT |
| | | CGGGTGCTATGGCTGCTCCAAGTGCAGGGAGGCATGGCGCCCGGCTG |
| | | GCGCTGCACAATCGGGAACCGCCCGCTGCTAGGCGTATGCGGACAGG |
| | | CGATTCTCTCGCGCCAAACGTGGCTTCTAGCGGCATTCTGGTAGCCGG |
| | | CTCTCGCGGTCGTCGGGCTTTTCAGAATCTGTCTTACTAACCTTCTCGA |
| | | AAGTATTGTCATGTCATGAGACAATACGGGAATGAAATGCAAACGGA |
| | | ACTCGGACGGTCGAGCGATCGATCATCATGCCCTTCAGGTGATGCGCC |
| | | AACAGGCGATCAAAGCAGTTCGTGAGGGTCAAACGGCGCAAAGCGTG |
| | | GCGGCGGCGCTGGGCGTGAATGTGCGAAGCGTCTTCAGGTGGCTTGC |
| | | CGATTATGCTAGCGGTGGCCAGCGTGCGCTGCTCGCCAAACCGATCCC |
| | | GGGGCGTCCGTCCAAAGTCAGCGGCGACGAGATGCGCTGGCTTGCCC |
| | | AAGCGGTGCGAGACAACACACCGCAGCAATACAAGTTCG |
| pCHC027 | SEQ ID NO: 61 | AGCTGCTACCTCGAGGCTGCACAAGAGATTCGAGCCGATTGCCATAA |
| (ΔpemK) | Upstream | CCCACTGCGACAGGTCACCTGGTAGGGGCTCAGCGTCGCGCCAAGTG |
| | | TGGCAGCCGTGGCGTCGGCCAGCGAAGAAAGCGCCCCGCGGCGGTGC |
| | | AGATTTCCGGCGGGTTGCCGAGAAAGGAGGCTCAAATGTCCTCACAG |
| | | GAAAACAGCGGCCATGTCGAACAAGGCAATGGCAACCGTGTCGAAGC |
| | | TGGCGCTAGCGGGGCTGCGGCATGGCGCCGGTGCGGTAGCCGGAGCC |
| | | CGGCGCGGCGCCAAGCAGATACGCAGGCTTCTGCCGGCAGACTCCTT |
| | | CGCGCGTTCGGCTGCGCAGCGCCGCTGCGCCGTCAGTCGTCACGCGG |
| | | GCAAATTTCATTTGTTGGCAGCGATGGCGAGCAACGCTCGAGGGAGC |
| | | GATGTAGATACGCGTTGAAACATGGATCTCTTATGTTTATACTTGTAT |
| | | CAACATTGTTTGGAGGCATCTATTATGCGAAAAAGCGCAACCCTGAC |
| | | GATTCAAAAGTGGGGCAACAGCTTGGCGGTTCGAATCCCCACTGCGG |
| | | TGGCTCGTTCTGCACATTTCGCCGAGGGCCAGGAAGTGGAGGTATCC |
| | | GTCGATGAGATTGGCGTAACTGTTCGACCAGTTGGTCGTCGTGCCCTC |
| | | ACTCTCGCGGAAAAGCTTGCTCTGTTTGACCCCATCAAGCACGGCGGC |
| | | GAAGCTATGGCCACGCAGCGTGTCGGCGCGGAGGCCATGTAA |
| | SEQ ID NO: 62 | CCTCCATGCTTGACCGCATGGCGCGTTGTCTTGGCGAACACGCCGAGC |
| | Downstream | CGTCGCAAGCTGGTCACTGCCAGCGCACACATGCGGCTCGACTGGAT |
| | | GAAGCCAGCCACGGTAGACGTAGACGTGATCAGCACATCAACCTGAC |
| | | CACCGAGCAGATCATTGATGGCCGACCCGGCGCCCTTGTACGGCACG |
| | | TGCTGCAGCGGCACGCTGCTGTTCTTGTCGAGCACAACGCCTATCAGG |
| | | TGCAACAGCGTGCCGATGCCGGGCGTGGCGTAGGTGATCTTCTGCAG |
| | | TTGTGCCTTGGCCTGTGTGGCCAGCGCCGGGCAGTGGGCGGGACTCGT |
| | | CGCGCCGCGCGGTCCGCGTGCCGAAGTCACTGCGCCGCCCAAGGTTC |
| | | GCGCTGCGGAACTGGGCGCCGGCCTGCGTGAGCGTCAATGCTGGCGG |
| | | CGGCCCCCCCGGCAGCATGGCGCGCCGCCAACCCCATGTTGAGGTTG |
| | | TGTCCCGGCGCTACTGGTCAACGCGCACAATCAACCTCACCTGGCGCA |
| | | CAACCTACCTCACCGATCCCTGCCGGCTGTTTCCTTCCAGCCTTTGTCC |
| | | AGCTTGGGAGATAAGACATATGCACAGGTCACGCACAATACATCTCA |
| | | CCTTAGGGGCATCAACACAACAAACCTCACCTTCTTGGGGCGGCTTCG |
| | | GATGCGGTGCCGTTCATCAGGCATCGTGTCCGCCGTAACGGGGATGTC |
| | | GATGTACAAACAGTAGTCGGCAAACACTATGCAAATA |
| pCHC030 | SEQ ID NO: 63 | CGAACTTGTATTGCTGCGGTGTGTTGTCTCGCACCGCTTGGGCAAGCC |
| (ΔCBBp | Upstream | AGCGCATCTCGTCGCCGCTGACTTTGGACGGACGCCCCGGGATCGGTT |
| ΔMBH | | TGGCGAGCAGCGCACGCTGGCCACCGCTAGCATAATCGGCAAGCCAC |
| ΔSH) | | CTGAAGACGCTTCGCACATTCACGCCCAGCGCCGCCGCCACGCTTTGC |
| | | GCCGTTTGACCCTCACGAACTGCTTTGATCGCCTGTTGGCGCATCACC |
| | | TGAAGGGCATGATGATCGATCGCTCGACCGTCCGAGTTCCGTTTGCAT |
| | | TTCATTCCCGTATTGTCTCATGACATGACAATACTTTCGAGAAGGTTA |
| | | GTAAGACAGATTCTGAAAAGCCCGACGACCGCGAGAGCCGGCTACCA |
| | | GAATGCCGCTAGAAGCCACGTTTGGCGCGAGAGAATCGCCTGTCCGC |
| | | ATACGCCTAGCAGCGGGCGGTTCCCGATTGTGCAGCGCCAGCCGGGC |
| | | GCCATGCCTCCCTGCACTTGGAGCAGCCATAGCACCCGAGCCGATCCC |
| | | ATGTACCACGCCACCGCCCCTTGCCTCCTCGCTATGCGGCACCGCCGA |
| | | AATGCTGCAAGCCAAGCTGTCCATGCTACTCCGCCAGTGCGTTGCACG |
| | | GCCGGGCACGCTACCGGTGACAGATGCCGACAAGGCACCTGCCATGA |
| | | GCGCACCCCAGCTGCGGACCACACTCTCGGTGATTCCAGCATGAGCA |
| | | AGGGGCCGGATGGCATGTTCGCTGACTACGTCTCGC |
| | SEQ ID NO: 64 | CGAGAGGGTAGAACATGTGCCTGGCCATACCTGCACGCATCGCGAAA |
| | Downstream | AAATTTGACAACGACATGGCCCTCATCGACCTGGGCGGCGTGGGGAG |
| | | TGGCCAAAAACGGGGGGCAAATCCGTCAGGAAAGGGGTCTATTGTGT |
| | | ACTGAGACTACCGGAGACCGCCATGCGCATCTCGATCCAAGCCTGTA |
| | | TTGAGCGGGCGGGCGAACAGCCCTCTAAGGTGATTGAAGTTGCGGTG |
| | | ATCGAGCGCAATGCCGATGTCGCTCCGGCCTCAGGACTGGGCCTGTTC |
| | | ATTCGCGAGTCACAAGAGATCCTGCGACAGCTTCAGACTGTGGTCTTG |
| | | ACCGAGCAGGTGGACCAGTTCATCCGGATTACCGGTCGCTGTCAACT |
| | | GTGCGGAGGCAGGCTTGTCATCAAGGACACAAAATCCTTGGTCTATC |
| | | GCACCGCTTTTGGCAAGGCGAGGCTGCGAAGCCCGCGCTTTTACTCTT |
| | | ACTGCAGCGCATGCGGTTACTGCTCAAGTAACAAGGGCACGCTTTCCC |
| | | CGCTGGCACAGGCGTTACCAGAACGCGTACATCCCCAGTGGACCTGG |
| | | CTGCAGTGCCGATATGCAAGCGTGATGTCTTATCGTTTGGCACAGATC |
| | | TTTCTACGCGACGCGTTTGCCGGCGGACGGGAACTCCCATGCTCGAGC |
| | | GTTAAGTTGAATGTAGGCCGGGTCGGGCAGCGGCTGGAGCAAGAGGC |
| | | GCAACGTGCAACGATGGTGATGTCGGCTGTGACCGCGCC |
| pCHC036 | SEQ ID NO: 65 | CGCCGTCGTCGCGAACTCGGTCTGCTGATCCTCAATCGCGAGCTTGCC |
| (ΔpHG1) | Upstream | GGCGCGGCAGTCGTTCCCGTGGCGCTGAAGCTCAATCACACGCGCCT |
| | | GCGCCCGCTCTTCGAGCAGTGGTGGCCATACATGAACCGCATGTCGCT |
| | | GAACCTGCAGCGCTTCGGGGCGGCTACTTTCTCGCGCACCGAACTGTC |
| | | GACGCTCGAGAACTACCTCGAACGCGAGCTCTCGAAGATCGAGGACT |
| | | ATGTGGACGAACAGCTGCGTGTGGCGAAAGCCTACCGCGAGCAACGG |
| | | GAACAGGAGATGCGAGCGAGAGGCGAGATCGTGTTCGTCCCGACGAT |
| | | CCAGCGCCCGTCCCTTGCGCTCGAGGTCCAGGCCTACTCGCGCTTTTC |
| | | CGTGCGTGCCCTGCAGGTTCTGATCAAGTTCGATCAAACCATGGACCA |
| | | GTTCGACTTTATGGTCTGGAACGGCATCCGTGACCAGAGCGACGTCA |
| | | ACGATGAAGTCACGCGCTTCCTACGCAAGTTCCAGCCGCTGGGCCTGC |
| | | GCAGCTACACCACTCACCTGAGGTTGATGACGACGGTGCGCTGTATTT |
| | | GACCACAGGAATTGATGTGACATGGTGTCACTTGGTGTGAGCTGGTG |
| | | AGACCACGTGGGACATGATCTCACTTCCGAACGAATCAATAGGCGTC |
| | | GACATCTGCCCCGAGGTACCACTTCGGGGCACTTGCTGTTTTGAACCG |
| | | GCACCAAGTGCCACTTACTGCCACACTGCGATCCGCC |
| | SEQ ID NO: 66 | GCGTTGGCGCCGATCCGATTGCACGCTCGCCTGCTGACCTACCTCTCT |
| | Downstream | GAGGTGAGCAACATGACCGCTCCGCTCCTGTTGGTTGAGATCGTCTCG |
| | | GAACGGCGCGTTAACAAAAGCACTATCGCCTGCCCAAACGACTCACG |
| | | GCTTTGCTGCCAACAGGAGACCCCCCAGATGAATAACGCTTCCGAGT |
| | | AAGTCACTGGAAGGCTTCGTGTTCCGACTCGGAACACCGAAGCTCAA |
| | | AGGTCTGAAGCAGCATCACCCGACTAAGGCGGGTCGTGCGGGTGTCC |
| | | GTGTTCTGACTTGGAACGCAGCTGAGTTTGTCCCGGCGATACCTCGGG |
| | | CGAGCGCATTATGGGCGTCAATGGAGGTCGCTTACGGGGACCCTTGT |
| | | AGGGCAGGCACTGCAAACGGACAGCAGCAATAGCTTTGCGGCGCAAG |
| | | ATCGCGCTGTTATGGAGCGCTGACGGCCGAGGACACGTCGCCTGAAA |
| | | GAGTGCGACATGATAAAGGCGGGCGCCGAGCGCCGGCTCTCATAGGG |
| | | GGACCGAGTTTGGCATCCGTGGCCACACCTCTCAATGAGTTGCCTTAC |
| | | GTCACCCAGAGCTTGCATGCCCCGGATTCGTCTCGTCTCAACTTGCGA |
| | | CATACCCTCGCGGGATCCCGCGACACGATGGCGCAAGGACCGACAGC |
| | | AAGGTCACAAAGTTTGTACGAATCCTAGTTGCAAGGCCTCCGTAAGC |
| | | CCTGTGCGCGGACTTGTTTAGCGCCGGACTCTGCCGCTAA |
| pCHC039 | SEQ ID NO: 67 | ACGACTTCGCCAAGGAACAGGTCGTAGGTCTGCTGCGTGGCCGGCTC |
| (ΔphcA) | Upstream | CGGCAGCAGCCGGCATTCCAGCCAGGCCGCGCAGCCCTCCAGCAGCG |
| | | GCGCGCCCACCGCCGTGCCGGCAAAGGTGCCAAGGCCGTAGGCGTCG |
| | | AACTTGTCGGTGCCTTCCTGCTCCATCAGCGCCAGGCCCGAGCTGGAG |
| | | CCCAGTGCCTCGGTCAGGTCGACCTGGCTGACGGTGGGGACCTGCAA |
| | | CACGAACTCGCCGCTGTCTTCCAGCAGGTGCCGGGTCCAGGTGCTCTT |
| | | GTCCAGCACCACTGCCACCTTGGGCGGGGCGAAGTCGAGCGGCATGG |
| | | CCCAGGCGGCGGCCATGATATTGCGCTTGCCGCCGGCGGCGGCGCTG |
| | | ACCAGCACAGTGGGGCCGTGGTTCAGCAAGCGGTAGGCTTTCGGGAG |
| | | TGATACGGGCAGGCGGAAATGTTCAGGCATGATGGCCGGGATGAGCC |
| | | GTCAGAAAAGAATGATAAAAATGGGAACGGCGGACCCACTATACCCG |
| | | GATGTACGAGTGCATGTTGCGGCGCGGGAAATGTTCACATATGCGGT |
| | | CAATTGTGGAAAAAGAGCGCAATTTTTCAGAAATATGGCGTAGACGG |
| | | CCATTTCAGAAATGCCGAATTTGCTTTCCGAGCTTGTTTTTTCCTCTTA |
| | | CACTATTAAGACGCCGTTGAAATCTGATGTGCAGCCAGTGCAAGTGG |
| | | TGGGGCCATCTAGCTAAGAATAATCTGACCGAGGCCTGATC |
| | SEQ ID NO: 68 | GCAGCATCCTGCGGCGAGCAGCCCAAACAAAAAACCGGCGCCTGGCG |
| | Downstream | CCGGTTTTTTGTTGCCCGTCTGCGCTCCGCGGTGGAGCGTGCAGGCTT |
| | | ATCGTTTGGGTCTGTGGGGACAGTCTGTTTTGGTGCAATTGCCGTACA |
| | | GCGACAGTGCATGTTCCTGCAGCGTAAAGCCGCGCTCGCGCGCGATG |
| | | CTTTGCTGGCGGTGCTCGATTTCAGAGTCGAAGAACTCCTCGACGCGG |
| | | CCGCAGTCGAGGCACACCAGGTGGTCATGGTGCTTGCCTTCGTTGAGT |
| | | TCGAAAATCGCCTTGCCGGATTCAAAGTTGTTGCGCGAGAGCAGGCC |
| | | CGCCTGCTCGAACTGGGTCAGCACGCGGTAGACGGTGGCCAGGCCGA |
| | | TGTCCATATGCTCGTTCAGCAGGATACGGTAGACGTCTTCCGCGCTCA |
| | | GGTGCCGCTGCTCACTGGTCTGAAAAATTTCAAGAATCTTCAGCCTGG |
| | | GCACGGTCGCCTTCAGGCCGATGTTCTTGAGGTCCGCCGGACTCGGCA |
| | | TGTGGGTGACTCCCTAGAGTACAATGACTGGATAGTTGAATCATAAG |
| | | GGTTTTGGCAGCAAAAGTCGCTCGCGGTAGTGATGTCCCAGGTCGAC |
| | | GTCACGCGCCCGGCACGGTTGCCGTCCCATGTGGTTGTCGCGGCAGGC |
| | | GCAACAAGGCGTGTGTGGTGCAATCGGCGCATATTGCGCCGTTTTTGT |
| | | GCCCGCTTTGCGGTACCGTGCCGCGGTACTTTTCTT |

Strain Construction.

To improve transformation efficiency by homologous recombination, the native Type 1 restriction enzyme (RE) defense system of C. necator was inhibited by deleting a restriction enzyme subunit (ΔH16_A0006), as described previously. All engineered strains were then derived from this restriction-deficient parental strain, CHC020 (H16 ΔRE).

Electrocompetent C. necator cells were prepared using a previously described optimized electroporation protocol. Competent cells were transformed with 1.5 to 4 μg plasmid DNA, using a Gene Pulser Xcell (Bio Rad) electroporator. The recovery period was conducted in 15 mL culture tubes with 900 μL SOC (New England Biolabs) for 2 h at 30° C. and 225 rpm. Transformants were selected by plating on LB agar plates with 200 μg/mL kanamycin (Kan:200), followed by outgrowth at 30° C. for 48 to 72 h. Transformations by conjugation were performed using E. coli S17-1 as the donor strain. Transformants were selected by plating on LB agar plates with 200 μg/mL kanamycin and 15 μg/mL gentamycin, followed by outgrowth at 30° C. for 48-72 h. Transformants were restruck on Kan:200 plates two additional times to ensure modifications were propagated throughout all copies of the genome.

Gene deletions were performed as described previously, with minor modifications. Typically, about 10 kanamycin-resistant transformant colonies from 3rd Kan:200 plates were picked and restruck on 15% sucrose YTS plates for SacB-mediated counter-selection. YTS plates contained 5 g/L yeast extract, 10 g/L tryptone, 15 g/L agar, and 150 g/L sucrose. After outgrowth for 72 h at 30° C., a first round of colony PCR genotyping was conducted with primers that anneal outside of the targeted homology regions. Colonies containing the expected deletions were then restruck on another YTS plate. After an additional 72 h, colonies from 2nd YTS plates were screened a second time, using primers interior to the targeted region, to confirm loss of the expected gene(s). Strains used in this study are described in Table 1, and all construction details are provided in Table 5.

TABLE 5
Construction details for strains disclosed herein.

| Strain | Genotype | Alias | Construction Details |
|---|---|---|---|
| CHC001 | Cupriavidus necator ATCC 17699 | H16 | Obtained from ATCC culture collection. |
| CHC004 | Cupriavidus necator DSM 542 | G + 7 | Obtained from DSM culture collection. |
| CHC020 | C. necator ATCC 17699 | H16 ΔRE | The restriction enzyme subunit H16_0006 was deleted from wild- |
| | ΔH16_A0006 | | type H16 by transforming strain CHC001 with pCHC005 by |
| | | | conjugation with E. coli S17-1. Following selection on |
| | | | LB + Kan: 200 + Gent: 15, isolated colonies were cured using sucrose |
| | | | selection. Candidates were screened using colony PCR, and gene |
| | | | deletion was confirmed by amplification of a 2423 bp product using |
| | | | primers oCHC031 and oCHC032. |
| CHC023 | C. necator ATCC 17699 | H16 ΔRE | The polyhydroxyalkanoate synthesis operon phaCAB was deleted |
| | ΔH16_A0006 | ΔphaCAB | from strain CHC020 (H16 ΔH16_A0006) by electroporation with |
| | ΔphaCAB | | plasmid pCHC004. Following selection on two rounds of |
| | | | LB + Kan: 250 plates, isolated colonies were cured using two rounds |
| | | | of sucrose counterselection on YTS plates. Candidates were |
| | | | screened using colony PCR, and phaCAB knockout was confirmed |
| | | | by amplification of a 1,788 bp product using primers oCHC224 and |
| | | | oCHC227. Deletion was further confirmed by the absence of a |
| | | | wildtype colony PCR product using primer pairs |
| | | | oCHC224/oCHC225 and oCHC226/oCHC227. |
| CHC045 | C. necator ATCC 17699, | ALE | A single colony of CHC001 designated “HA” was selected for |
| | Formate ALE, | HA6 | adaptive laboratory evolution. The colony was inoculated into 5 mL |
| | Population A, | | of MSM medium containing 50 mM sodium formate. Cultures were |
| | Generation 400, | | repeatedly grown until saturation, after approximately 24 hours, at |
| | Colony #6 | | which point they were restarted by reinoculation of 100-200 μL of |
| | | | cells intro fresh media. Serial subculturing was continued for about |
| | | | 400 generations, after which ALE was terminated. The final evolved |
| | | | HA population was struck out on an LB plate to generate isolated |
| | | | colonies for screening. Using a 96-well plate reader, colony HA6 |
| | | | was determined to be a top performing strain based upon its |
| | | | improved growth rate on formate. |
| CHC046 | C. necator ATCC 17699, | ALE | A single colony of CHC001 designated “HB” was selected for |
| | Formate ALE, | HB3 | adaptive laboratory evolution. The colony was inoculated into 5 mL |
| | Population B, | | of MSM medium containing 50 mM sodium formate. Cultures were |
| | Generation 400, | | repeatedly grown until saturation, after approximately 24 hours, at |
| | Colony #3 | | which point they were restarted by reinoculation of 100-200 μL of |
| | | | cells intro fresh media. Serial subculturing was continued for about |
| | | | 400 generations, after which ALE was terminated. The final evolved |
| | | | HB population was struck out on an LB plate to generate isolated |
| | | | colonies for screening. Using a 96-well plate reader, colony HB3 |
| | | | was determined to be a top performing strain based upon its |
| | | | improved growth rate on formate. |
| CHC048 | C. necator ATCC 17699, | ALE | A single colony of CHC001 designated “HC” was selected for |
| | Formate ALE, | HC8 | adaptive laboratory evolution. The colony was inoculated into 5 mL |
| | Population C, | | of MSM medium containing 50 mM sodium formate. Cultures were |
| | Generation 400, | | repeatedly grown until saturation, after approximately 24 hours, at |
| | Colony #8 | | which point they were restarted by reinoculation of 100-200 μL of |
| | | | cells intro fresh media. Serial subculturing was continued for about |
| | | | 400 generations, after which ALE was terminated. The final evolved |
| | | | HC population was struck out on an LB plate to generate isolated |
| | | | colonies for screening. Using a 96-well plate reader, colony HC8 |
| | | | was determined to be a top performing strain based upon its |
| | | | improved growth rate on formate. |
| CHC050 | C. necator DSM 542, | ALE | A single colony of CHC004 designated “GD” was selected for |
| | Formate ALE, | GD2 | adaptive laboratory evolution. The colony was inoculated into 5 mL |
| | Population D, | | of MSM medium containing 50 mM sodium formate. Cultures were |
| | Generation 400, | | repeatedly grown until saturation, after approximately 24 hours, at |
| | Colony #2 | | which point they were restarted by reinoculation of 100-200 μL of |
| | | | cells intro fresh media. Serial subculturing was continued for about |
| | | | 400 generations, after which ALE was terminated. The final evolved |
| | | | GD population was struck out on an LB plate to generate isolated |
| | | | colonies for screening. Using a 96-well plate reader, colony GD2 |
| | | | was determined to be a top performing strain based upon its |
| | | | improved growth rate on formate. |
| CHC053 | C. necator DSM 542, | ALE GE7 | A single colony of CHC004 designated “GE” was selected for |
| | Formate ALE, | | adaptive laboratory evolution. The colony was inoculated into 5 mL |
| | Population E, | | of MSM medium containing 50 mM sodium formate. Cultures were |
| | Generation 400, | | repeatedly grown until saturation, after approximately 24 hours, at |
| | Colony #7 | | which point they were restarted by reinoculation of 100-200 μL of |
| | | | cells intro fresh media. Serial subculturing was continued for about |
| | | | 400 generations, after which ALE was terminated. The final evolved |
| | | | GE population was struck out on an LB plate to generate isolated |
| | | | colonies for screening. Using a 96-well plate reader, colony GE7 was |
| | | | determined to be a top performing strain based upon its improved |
| | | | growth rate on formate. |
| CHC055 | C. necator DSM 542, | ALE GF4 | A single colony of CHC004 designated “GF” was selected for |
| | Formate ALE, | | adaptive laboratory evolution. The colony was inoculated into 5 mL |
| | Population F, | | of MSM medium containing 50 mM sodium formate. Cultures were |
| | Generation 400, | | repeatedly grown until saturation, after approximately 24 hours, at |
| | Colony #4 | | which point they were restarted by reinoculation of 100-200 μL of |
| | | | cells intro fresh media. Serial subculturing was continued for about |
| | | | 400 generations, after which ALE was terminated. The final evolved |
| | | | GF population was struck out on an LB plate to generate isolated |
| | | | colonies for screening. Using a 96-well plate reader, colony GF4 was |
| | | | determined to be a top performing strain based upon its improved |
| | | | growth rate on formate. |
| CHC076 | C. necator ATCC 17699 | H16 ΔRE | The transcriptional regulator gene phcA was deleted from strain |
| | ΔH16_A0006 | ΔphcA | CHC020 (H16 ΔRE) by electroporation with plasmid pCHC022. |
| | ΔphcA | | Following selection on three rounds of LB + Kan: 250 plates, isolated |
| | | | colonies were cured using two rounds of sucrose counterselection on |
| | | | YTS plates. Candidates were screened using colony PCR, and phcA |
| | | | knockout was confirmed by amplification of a 1,743 bp product |
| | | | using primers oCHC142 and oCHC143. Deletion was further |
| | | | confirmed by the absence of a wildtype colony PCR product using |
| | | | primers oCHC163 and oCHC143. |
| CHC077 | C. necator ATCC 17699 | H16 ΔRE | The membrane-bound hydrogenase operon was deleted from strain |
| | ΔH16_A0006 | ΔMBH | CHC020 (H16 ΔRE) by electroporation with plasmid pCHC023. |
| | ΔhoxKGZMLOQRTV | | Following selection on three rounds of LB + Kan: 250 plates, isolated |
| | ΔhypA1B1F1CDEX | | colonies were cured using two rounds of sucrose counterselection on |
| | ΔhoxABCJ | | YTS plates. Candidates were screened using colony PCR, and MBH |
| | | | knockout was confirmed by amplification of a 1,809 bp product |
| | | | using primers oCHC150 and oCHC151. Deletion was further |
| | | | confirmed by the absence of a wildtype colony PCR product using |
| | | | primers oCHC165 and oCHC151. |
| CHC078 | C. necator ATCC 17699 | H16 ΔRE | The soluble hydrogenase operon was deleted from strain CHC020 |
| | ΔH16_A0006 | ΔSH | (H16 ΔRE) by electroporation with plasmid pCHC024. Following |
| | ΔhoxFUYHWI | | selection on three rounds of LB + Kan: 250 plates, isolated colonies |
| | ΔhypA2B2F2 | | were cured using two rounds of sucrose counterselection on YTS |
| | | | plates. Candidates were screened using colony PCR, and SH |
| | | | knockout was confirmed by amplification of a 1,787 bp product |
| | | | using primers oCHC148 and oCHC149. Deletion was further |
| | | | confirmed by the absence of wildtype colony PCR products using |
| | | | primer pairs oCHC148/168 and oCHC167/149. |
| CHC079 | C. necator ATCC 17699 | H16 ΔRE | The megaplasmid copy of the CBB operon was deleted from strain |
| | ΔH16_A0006 | ΔCBBp | CHC020 (H16 ΔRE) by electroporation with plasmid pCHC025. |
| | ΔcbbR′ | | Following selection on three rounds of LB + Kan: 250 plates, isolated |
| | ΔcbbLpSpXpYpEpFpPpTpZpGpKpAp | | colonies were cured using two rounds of sucrose counterselection on |
| | | | YTS plates. Candidates were screened using colony PCR, and CBBp |
| | | | knockout was confirmed by amplification of a 1,670 bp product |
| | | | using primers oCHC152 and oCHC153. Deletion was further |
| | | | confirmed by the absence of wildtype colony PCR products using |
| | | | primer pairs oCHC152/170 and oCHC179/153. |
| CHC081 | C. necator ATCC 17699 | H16 ΔRE | The plasmid addiction system toxin PemK was deleted from strain |
| | ΔH16_A0006 | ΔpemK | CHC020 (H16 ΔRE) by conjugation with plasmid pCHC027. |
| | ΔpemK | | Following selection on LB + Kan: 200 + Gent: 15, and two round of |
| | | | selection on LB + Kan: 250 plates, isolated colonies were cured using |
| | | | two rounds of sucrose counterselection on YTS plates. Candidates |
| | | | were screened using colony PCR, and pemK knockout was |
| | | | confirmed by amplification of a 1,589 bp product using primers |
| | | | oCHC144 and oCHC145. Deletion was further confirmed by the |
| | | | absence of wildtype colony PCR products using primer pairs |
| | | | oCHC144/174 and oCHC173/145. |
| CHC092 | C. necator ATCC 17699 | H16 ΔRE | A 103,552 bp region of the megaplasmid (encompassing the CBBp, |
| | ΔH16_A0006 | ΔCBBp | MBH, SH operons and intervening sequences) was deleted from |
| | ΔcbbR′ | ΔMBH | strain CHC020 (H16 ΔRE) by electroporation with plasmid |
| | ΔcbbLpSpXpYpEpFpPpTpZpGpKpAp | ΔSH | pCHC030. Following selection on three rounds of LB + Kan: 250 |
| | ΔhoxKGZMLOQRTV | | plates, isolated colonies were cured using two rounds of sucrose |
| | ΔhypA1B1F1CDEX | | counterselection on YTS plates. Candidates were screened using |
| | ΔhoxABCJ | | colony PCR, and the knockout was confirmed by amplification of a |
| | ΔPHG023-087 | | 1,772 bp product using primers oCHC153 and oCHC149. Deletion |
| | ΔhoxFUYHWI | | was further confirmed by the absence of wildtype colony PCR |
| | ΔhypA2B2F2 | | products using primer pairs oCHC179/153, oCHC150/166, and |
| | | | oCHC167/149. |
| CHC099 | C. necator ATCC 17699 | H16 ΔRE | The transcriptional regulator gene phcA was deleted from strain |
| | ΔH16_A0006 | ΔCBBp | CHC092 (H16 ΔRE ΔCBBp ΔMBH ΔSH) by electroporation with |
| | ΔcbbR′ | ΔMBH | plasmid pCHC022. Following selection on three rounds of |
| | ΔcbbLpSpXpYpEpFpPpTpZpGpKpAp | ΔSH | LB + Kan: 250 plates, isolated colonies were cured using three rounds |
| | ΔhoxKGZMLOQRTV | ΔphcA | of sucrose counterselection on YTS plates. Candidates were |
| | ΔhypA1B1F1CDEX | | screened using colony PCR, and phcA knockout was confirmed by |
| | ΔhoxABCJ | | amplification of a 1,743 bp product using primers oCHC142 and |
| | ΔPHG023-087 | | oCHC143. Deletion was further confirmed by the absence of |
| | ΔhoxFUYHWI | | wildtype colony PCR products using primer pairs oCHC142/164 and |
| | ΔhypA2B2F3 ΔphcA | | oCHC163/143. |
| CHC105 | C. necator ATCC 17699 | H16 ΔRE | The entire pHG1 megaplasmid was deleted from strain CHC081 |
| | ΔH16_A0006 | ΔpHG1 | (H16 ΔRE ΔpemK) by conjugation with plasmid pCHC036. |
| | ΔpHG1 | | Following selection on LB + Kan: 200 + Gent: 15, and two round of |
| | | | selection on LB + Kan: 250 plates, isolated colonies were cured using |
| | | | three rounds of sucrose counterselection on YTS plates. |
| | | | Megaplasmid loss was first detectable by the presence of |
| | | | significantly fainter wildtype bands when 1st YTS colonies were |
| | | | screened using colony PCR with primers oCHC148 and oCHC168. |
| | | | Megaplasmid loss was further confirmed in 2nd YTS colonies by the |
| | | | absence of wildtype colony PCR products using primer pairs |
| | | | oCHC148/168, oCHC150/166, oCHC152/170, and oCHC171/147. |
| CHC113 | C. necator ATCC 17699 | H16 ΔRE | The transcriptional regulator gene phcA was deleted from strain |
| | ΔH16_A0006 | ΔpHG1 | CHC105 (H16 ΔRE ΔpHG1) by conjugation with plasmid |
| | ΔpHG1 | ΔphcA | pCHC039. Following selection on LB + Kan: 200 + Gent: 15, and two |
| | ΔphcA | | round of selection on LB + Kan: 250 plates, isolated colonies were |
| | | | cured using two rounds of sucrose counterselection on YTS plates. |
| | | | Candidates were screened using colony PCR, and phcA knockout |
| | | | was confirmed by amplification of a 1,743 bp product using primers |
| | | | oCHC142 and oCHC143. Deletion was further confirmed by the |
| | | | absence of wildtype colony PCR products using primer pairs |
| | | | oCHC142/164 and oCHC163/143. |
| CHC122 | C. necator ATCC 17699 | H16 ΔRE | The polyhydroxyalkanoate synthesis operon phaCAB was deleted |
| | ΔH16_A0006 | ΔphcA | from strain CHC076 (H16 ΔRE ΔphcA) by conjugation with plasmid |
| | ΔphcA | ΔphaCAB | pCHC042. Following selection on LB + Kan: 200 + Gent: 15, and two |
| | ΔphaCAB | | rounds of LB + Kan: 200 plates, isolated colonies were cured using |
| | | | two rounds of sucrose counterselection on YTS plates. Candidates |
| | | | were screened using colony PCR, and phaCAB knockout was |
| | | | confirmed by amplification of a 1,788 bp product using primers |
| | | | oCHC224 and oCHC227. Deletion was further confirmed by the |
| | | | absence of a wildtype colony PCR product using primer pairs |
| | | | oCHC224/oCHC225 and oCHC226/oCHC227. |
| CHC123 | C. necator ATCC 17699 | H16 ΔRE | The polyhydroxyalkanoate synthesis operon phaCAB was deleted |
| | ΔH16_A0006 | ΔpHG1 | from strain CHC105 (H16 ΔRE ΔpHG1) by conjugation with |
| | ΔpHG1 | ΔphaCAB | plasmid pCHC042. Following selection on LB + Kan: 200 + Gent: 15, |
| | ΔphaCAB | | and two rounds of LB + Kan: 200 plates, isolated colonies were cured |
| | | | using two rounds of sucrose counterselection on YTS plates. |
| | | | Candidates were screened using colony PCR, and phaCAB knockout |
| | | | was confirmed by amplification of a 1,788 bp product using primers |
| | | | oCHC224 and oCHC227. Deletion was further confirmed by the |
| | | | absence of a wildtype colony PCR product using primer pairs |
| | | | oCHC224/oCHC225 and oCHC226/oCHC227. |
| CHC124 | C. necator ATCC 17699 | H16 ΔRE | The polyhydroxyalkanoate synthesis operon phaCAB was deleted |
| | ΔH16_A0006 | ΔpHG1 | from strain CHC113 (H16 ΔRE ΔpHG1 ΔphcA) by conjugation with |
| | ΔpHG1 | ΔphcA | plasmid pCHC042. Following selection on LB + Kan: 200 + Gent: 15, |
| | ΔphcA | ΔphaCAB | and two rounds of LB + Kan: 200 plates, isolated colonies were cured |
| | ΔphaCAB | | using two rounds of sucrose counterselection on YTS plates. |
| | | | Candidates were screened using colony PCR, and phaCAB knockout |
| | | | was confirmed by amplification of a 1,788 bp product using primers |
| | | | oCHC224 and oCHC27. Deletion was further confirmed by the |
| | | | absence of a wildtype colony PCR product using primer pairs |
| | | | oCHC224/oCHC225 and oCHC226/oCHC227. |

Deletion of Megaplasmid pHG1.

Deletion of pHG1 was accomplished via two transformation steps using the strain construction methods outlined above, with some modifications. First, the megaplasmid addiction system was disrupted by deleting the toxin-encoding gene pemK in strain CHC020 (H16 ΔRE) via conjugation with plasmid pCHC027 (ΔpemK). In the resulting strain, CHC081 (H16 ΔRE ΔpemK), an additional conjugation was performed to delete the entire megaplasmid replication region, using plasmid pCHC036 (ΔpHG1). In the resulting transformants, colony PCR screening on 1st YTS plates was challenging, but putative positive colonies were identified by having fainter bands during genotyping. Upon restreaking these onto 2nd YTS plates, megaplasmid loss was confirmed by the absence of all colony PCR bands corresponding to the presence of the soluble hydrogenase (SH) operon, the membrane-bound hydrogenase (MBH) operon, the megaplasmid CBB operon (CBBp), and the megaplasmid partitioning system operon (parAB). Deletion of pHG1 was also demonstrated definitively by total loss of all megaplasmid transcripts in our RNA-seq datasets.

Media Composition.

Cells were cultivated in minimal salt media (MSM) containing 3.746 g/L K₂HPO₄, 1.156 g/L KH₂PO₄, 0.962 g/L NH₄Cl, 0.702 g/L NaCl, 66 mg/L citric acid, 16.68 mg/L FeSO₄.7H₂O, 0.1 mg/L ZnCl₂, 0.03 mg/L MnCl₂.4H₂O, 0.05 mg/L CoCl₂.6H₂O, 0.07 mg/L CuCl₂.2H₂O, 0.12 mg/L NiCl₂.6H₂O, 0.03 mg/L Na₂MoO₄.2H₂O, 0.05 mg/L CrCl₃.6H₂O, 0.3 mg/L H₃BO₃, 11 mg/L CaCl₂, and 240 mg/L MgSO₄. Growth on formate was conducted in MSM media supplemented with 50 mM of sodium formate, unless otherwise indicated. Growth on alternate carbon sources was conducted in MSM media supplemented with either: 42 mM sodium acetate, 12 mM sodium benzoate, 21 mM sodium succinate, or 14 mM fructose.

Adaptive Laboratory Evolution.

To begin, wild-type C. necator (H16, ATCC 17699) and the glucose-utilizing C. necator mutant G+7 (DSM 542) were revived from glycerol stocks. Three isolated H16 colonies (designated HA, HB, and HC) and three G+7 colonies (GD, GE, and GF) were selected for parallel adaptive laboratory evolution. Each colony was inoculated into a 16×100 mm glass tube containing 5 mL of MSM with 50 mM of sodium formate and cultivated overnight at 30° C. and 225 rpm. Serial passaging into fresh media was repeated once every about 24 h, with initial and final optical density readings at 600 nm (OD₆₀₀) recorded by a Spectronic 601 spectrophotometer. The number of generations per day was calculated using the formula: # generations=ln (OD_{final}/ODᵢₙᵢₜᵢₐₗ)/ln(2). The reinoculation volume was initially 250 μL (5% of the culture volume) but was gradually reduced to 100 μL (2%) as growth rates improved. ALE was paused as needed by temporarily placing cultures in a refrigerator at 4° C. for up to 2 days, or by restarting from archived glycerol stocks. Adaptive laboratory evolution was continued until each lineage had reached a total of 400 generations. Performance of evolved populations was assayed by isolating individual colonies from each lineage and measuring their growth on MSM with 50 mM sodium formate using a microplate reader.

Microplate Reader Evaluation.

Strains were revived from glycerol stocks on LB plates, and then grown in test media until growing exponentially, at which point cultures were reinoculated into fresh media with variable volumes to normalize cultures to equal initial OD₆₀₀ values. For experiments evaluating growth on formate, strains were cultivated in 100-well honeycomb microplates with 200 μL of cells per well, tested in quadruplicate. Growth was measured using a Bioscreen C Pro microplate reader (Growth Curves USA), incubated at 30° C. under continuous orbital shaking at maximum amplitude, with absorbance readings at 600 nm taken every 15 minutes over 36 hours. For experiments evaluating growth on acetate, benzoate, fructose, and succinate, strains were cultivated in 48-well FlowerPlates (MTP-48-BOH2, m2p-labs) covered with gas-permeable sealing foil (F-GPR48-10, m2p-labs), with 900 μL, of cells per well, tested in triplicate. Growth was measured using a BioLector II microtiter plate reader (m2p-labs), incubated at 30° C. and 1300 rpm, with readings taken every 12 minutes over 48 hours. Data generated by microplate readers was analyzed using the GrowthRates software tool (Bellingham Research Institute), to calculate the maximum growth rate (μMax) of each strain. GrowthRates determines μMax by plotting ln(OD₆₀₀) versus time for each replicate, and identifying the maximum slope of a best fit trend line incorporating at least 5 data points. For each condition, the μMax values of biological replicates were compared using the two-sample t-test to determine whether differences in maximum growth rates were statistically significant (p≤0.05) compared to the wildtype.

Whole Genome Sequencing.

The best performing evolved isolate from each ALE lineage (designated HA6, HB3, HC8, GD2, GE7, and GF4) and their respective parental strains (HAT0, HBT0, HCT0, GDT0, GET0, GFT0) were chosen for whole genome sequencing. Genomic DNA was extracted from each strain using a Quick-DNA Fungal/Bacterial Miniprep Kit (Zymo Research). Purified genomic DNA was submitted to GENEWIZ, Inc. for sample QC, library preparation, and sequencing. Genomic DNA libraries were prepared using TruSeq Paired-End Sequencing Kits (Illumina, Inc.), and sequencing was completed using the Illumina MiSeq platform with a 2×150 bp configuration. Raw FASTQ data (about 1.1 million paired reads per sample) was then aligned to previously published reference genomes for chromosome 1 (NCBI NC_008313.1), chromosome 2 (NCBI NC_008314.1), and the megaplasmid pHG1 (NCBI NC_005241.1) using the Illumina DRAGEN pipeline. Next, we analyzed alignment files using the Geneious Prime bioinformatics software platform, version 2020.2.5 (Biomatters Ltd). Comparison of the parental and evolved isolates was completed using the Geneious SNP/INDEL variant finder (minimum coverage: 9 reads, minimum variant frequency: 67%) to identify locations which differed from the reference genomes.

RNA-Seq Transcriptomics.

Strains were revived from glycerol stocks on LB plates, and then grown for 15 to 20 h in MSM+2 g/L fructose (FN) or MSM+40 mM sodium formate+10 mM formic acid (MSMF) media at 30° C. and 225 rpm. Overnight cultures were then reinoculated into triplicate 250 mL baffled flasks containing a total of 50 mL FN or MSMF media at an initial OD₆₀₀ of about 0.07. Flasks were grown at 30° C. and 225 rpm for 12 h, with OD₆₀₀ readings taken every 1-1.5 h. Samples for RNA-seq analysis were taken once cultures reached a mid-log growth phase, at an OD₆₀₀ of about 0.85 for FN cultures or an OD₆₀₀ of about 0.30 for MSMF cultures. Cells were harvested by removal and centrifugation of 2 mL (FN) or 10 mL (MSMF) from each flask at 15,000 rpm for 1 minute. Following centrifugation, the supernatant was discarded, and cell pellets were immediately flash frozen in liquid nitrogen and stored at −80° C. until analysis. Samples were submitted on dry ice to GENEWIZ, Inc. for RNA extraction, QC, rRNA depletion, and library preparation. RNA-seq was completed using the Illumina, Inc. HiSeq platform with 2×150 bp configuration. Raw FASTQ data (about 23.6 million reads per sample) was then aligned to previously published reference genomes using the Geneious Prime software platform, version 2020.2.5 (Biomatters Ltd). Geneious was used to calculate expression levels for every gene in the genome, normalized by total transcript count for each sample, and reported as transcripts per million (TPM). For comparisons of global expression levels between strains and/or conditions, triplicate samples were grouped together and compared using the DESeq2 method. All differential expression analyses are included in SI File 3. Geneious DESeq2 outputs include Log₂ ratios, p-values, PCA Plots, and Volcano Plots.

Bioreactor Cultivations.

Strains were revived from glycerol stocks on LB plates, and then grown for 15 h at 30° C. and 225 rpm in triplicate 250 mL baffled flasks containing 50 mL of a 50:50 (v/v) mixture of MSM with 10 g/L fructose and LB. Overnight cultures were centrifuged at 4,000 rpm for 10 minutes and resuspended in MSM with 20 mM sodium formate to normalize OD₆₀₀ values to 5.0. Next, 30 mL of each culture were transferred to 250 mL flasks and supplemented with 1 mL LB, for a 6 h adaptation at 30° C. and 225 rpm. Adapted cultures were inoculated in bioreactors as biological triplicates, with the exception of strain CHC122, which was analyzed in duplicate due to a failed cultivation of the third replicate. Cultivations were carried out at 30° C. in 500 mL bioreactors (BioStat-Q Plus, Sartorius, Goettingen, Germany) containing 250 mL of MSM with 20 mM sodium formate, inoculated at an initial OD₆₀₀ of 0.1. Aerobic conditions were maintained with continuous sparging of air at 1 vvm, and the dissolved oxygen level was set at 25% by automated adjusting of the agitation speed between 350 and 1200 rpm. A pH-stat fed batch mode was used, where pH was maintained at 6.7 by the addition of a feed solution consisting of 35% formic acid (w/v) and 250 mM NH₃(aq) in modified MSM media containing 3× the standard concentrations of FeSO₄.7H₂O, ZnCl₂, MnCl₂.4H₂O, CoCl₂.6H₂O, CuCl₂.2H₂O, NiCl₂.6H₂O, Na₂MoO₄.2H₂O, CrCl₃.6H₂O, and H₃BO₃. To monitor growth, reactors were sampled every 2 hours for OD₆₀₀ and HPLC measurements until 200 mL of feed was exhausted. At the point of feed exhaustion, 50 mL of culture was sampled from each bioreactor. Samples were centrifugated and cell pellets were freeze dried by lyophilization for determination of total cell dry weight (CDW) and polyhydroxyalkanoate (PHA) content. Formic acid and cultivation co-products (pyruvic acid, acetic acid, lactic acid, succinic acid, and glycerol) were analyzed as with a modified injection volume of 6 μL and mobile phase of 0.02N H₂SO₄ to enable baseline separation of pyruvic acid and succinic acid from other analytes of interest. For each strain, maximum growth rate (μMax) values were calculated using GrowthRates, as described above for microplate reader experiments. Differences were calculated in comparison to the CHC023 (ΔphaCAB) control strain, using the two-sample t-test with a p-value of less than or equal to 0.20 to account for greater variation inherent to bioreactor cultivation.

Results

Adaptive laboratory evolution and whole genome sequencing reveal targets for improving formatotrophy.

Cupriavidus necator is a metabolic generalist, capable of adapting to variable resources and dynamic conditions and, consequently, it is likely not optimized for growth on formate alone. Therefore, we hypothesized that its growth on formate could be improved upon using ALE.

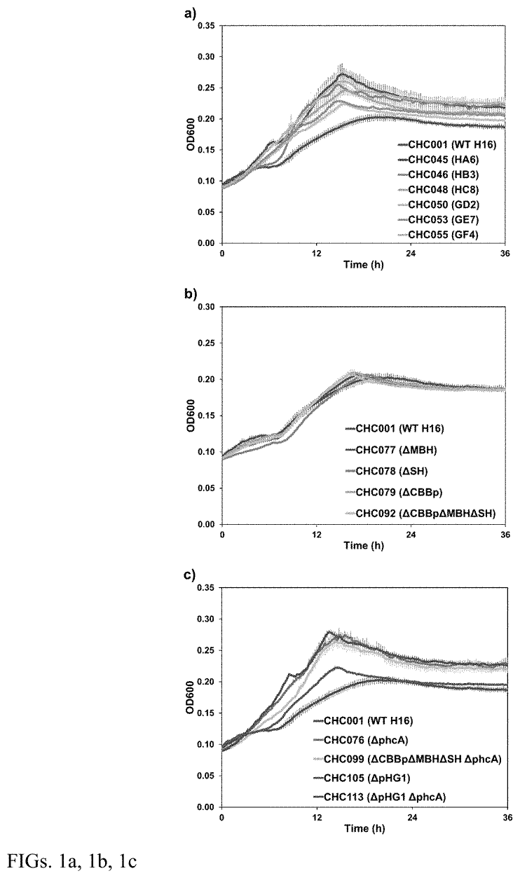

In order to select for random genetic mutations that improve growth on formate, we performed ALE of C. necator in six separate lineages grown in parallel on minimal medium containing 50 mM sodium formate as the source of carbon and energy. A concentration of 50 mM was chosen to maximize the amount of carbon available for growth, while minimizing the growth inhibition observed at higher formate concentrations. Three lineages were performed using C. necator H16 and three were performed with C. necator G+7, a previously isolated mutant of H16 capable of growing on glucose. ALE was conducted by serial transfer of cultures roughly every 24 hours, after reaching stationary phase. Following 400 generations of evolution, we isolated and evaluated the growth of ten individual colonies from each of the six populations and selected the best performing isolate from each lineage of H16 (designated HA6, HB3, HC8) and G+7 (GD2, GE7, and GF4) for further evaluation. These evolved isolates substantially outperformed wild-type C. necator when grown on minimal media with 50 mM sodium formate, exhibiting 1.15× to 2.18× faster maximum growth rates, as well as 10% to 34% greater maximum optical density at 600 nm (OD₆₀₀) under these conditions (FIG. 1a, Table 6).

TABLE 6
Maximum growth rates (μMax) on 200 μL formate microplates.

| | Strain | | | | | | |
|---|---|---|---|---|---|---|---|
| | CHC 001 | CHC 004 | CHC 045 | CHC 046 | CHC 048 | CHC 050 | CHC 053 |
| | Alias | | | | | | |
| | WT | | ALE | ALE | ALE | ALE | ALE |
| | H16 | G + 7 | HA6 | HB3 | HC8 | GD2 | GE7 |
| μMax | 0.080 ± 0.001 | 0.061 ± 0.005 | 0.112* ± 0.005 | 0.174* ± 0.019 | 0.111* ± 0.025 | 0.092* ± 0.003 | 0.099* ± 0.002 |

| | Strain | | | | | |
|---|---|---|---|---|---|---|
| | CHC 055 | CHC 077 | CHC 078 | CHC 079 | CHC 092 | CHC 099 |
| | Alias | | | | | |
| | | | | | | ΔCBBp |
| | | | | | ΔCBBp | ΔMBH |
| | ALE | | | | ΔMBH | ΔSH |
| | GF4 | ΔMBH | ΔSH | ΔCBBp | ΔSH | ΔphcA |
| μMax | 0.114* ± 0.011 | 0.096* ± 0.011 | 0.100* ± 0.003 | 0.093* ± 0.002 | 0.094* ± 0.004 | 0.110* ± 0.012 |

Values with an asterisk indicate a statistically significant (p≤0.05) increase in μMax, compared to the wildtype grown under the same conditions

To elucidate the nature of mutations that improved growth on formate in these isolates, we completed whole genome sequencing of each, as well as their unevolved parents. By comparing the genomes of the parental strains (HA, HB, HC, GD, GE, GF) to their corresponding evolved descendants (HA6, HB3, HC8, GD2, GE7, and GF4, respectively), we were able to identify mutations that had arisen in each lineage. We detected 147 SNPs or INDELs unrelated to ALE, including 5 unique to all G+7 strains, that represent differences between our lab strain of C. necator H16 and the published reference genomes. In addition, we found several mutations that were present in our evolved strains but not found in any parental strains, which could implicate them in improving growth on formate.

In some cases, SNPs were found in only one or two of the evolved strains. Strain HC8 contained a mutation in a subunit of an RNA polymerase, and strains HA6 and HB3 possessed a mutation in the transcription termination factor Rho. Mutations such as these, which can impact the expression of many genes, are often found in ALE experiments. We also found several interesting mutations that were localized to the same regions in multiple isolates, irrespective of whether they were derived from H16 or G+7. We focused our attention on those mutations, since similar mutations that converged in multiple independent ALE lineages were most likely to be responsible for the observed improvements in formatotrophic growth. These mutations are summarized in Table 7.

TABLE 7
An embodiment of mutations found in sequenced
strains obtained from formate ALE.

| | Megaplasmid pHG1 | | | | |
|---|---|---|---|---|---|
| | Calvin- | Membrane | | Total | Chromosome 1 |
| | Benson- | Bound | Soluble | pHG1 | Transcriptional |
| ALE | Bassham | Hydrogenase | Hydrogenase | Deletion | Regulator |
| Isolate | (CBBp) | (MBH) | (SH) | (kbp) | phcA |
| HA6 | Wildtype | Wildtype | Wildtype | 0 | INDEL |
| | sequence | sequence | sequence | | (Frameshift) |
| HB3 | Partial Δ | Total Δ | Wildtype | 42 | INDEL |
| | | | | | (Frameshift) |
| HC8 | Total Δ | Total Δ | Total Δ | 124 | Wildtype |
| | | | | | sequence |
| GD2 | Total Δ | Total Δ | Partial Δ | 121 | INDEL |
| | | | | | (Frameshift) |
| GE7 | Total Δ | Total Δ | Partial Δ | 121 | INDEL |
| | | | | | (Frameshift) |

| | | | | |
|---|---|---|---|---|
| GF4 | Total Δ | hoxA SNP (Substitution) | 12 | Wildtype |
| | | | | sequence |

For example, we found that four evolved strains (HA6, HB3, GD2, GE7) all obtained insertion or deletion mutations that lead to a frameshift in phcA, which encodes a LysR family transcriptional regulator. Furthermore, in five out of the six evolved strains (HB3, HC8, GD2, GE7, GF4) we discovered large deletions in the genome (ranging from 12 to 124 kbp) that were all localized to the same region of the megaplasmid pHG1. The deleted regions encompassed three major gene clusters: the membrane-bound hydrogenase complex (MBH; found in 4 of 6 strains), the soluble hydrogenase complex (SH; found in 3 of 6 strains), and, surprisingly, the pHG1 copy of the Calvin-Benson-Bassham cycle operon (CBBp; found in 5 of 6 strains). The evolved isolate GF4 contained a mutation in the regulator HoxA, which controls expression of both the MBH and the SH. Note that the CBBp, MBH, and SH clusters are located adjacent to one another on pHG1, such that the deletions summarized in Table 7 represent a single contiguous region of the megaplasmid in each strain.

Improved performance of evolved strains can be reconstituted by ALE-inspired metabolic engineering.

We next sought to recapitulate the improved performance of our evolved strains by systematically investigating the effect of reintroducing a series of ALE-inspired mutations into a wild-type background. The resulting strains (Table 1) contained complete genomic deletions of the transcriptional regulator PhcA (ΔphcA), the membrane-bound hydrogenase operon (ΔMBH), the soluble hydrogenase operon (ΔSH), the megaplasmid CBB operon (ΔCBBp), the combined 103,552 bp region spanning all three operons and intervening sequences (ΔCBBp ΔMBH ΔSH), or a combination of multiple deletions (ΔCBBp ΔMBH ΔSH ΔphcA). Given the prevalence of large genomic deletions on the megaplasmid in ALE strains, we hypothesized that pHG1, which accounts for 6.1% of the genome, might be dispensable for growth on formate. To examine this, the entire megaplasmid was eliminated via a two-step knockout strategy. First, we deleted the megaplasmid addiction gene pemK, which is a member of the pemIK anti-toxin/toxin system that ensures all progeny must receive a copy of pHG1 during cell division in order to survive. With pemK eliminated, we then deleted a 9.0 kb region of pHG1 that contains several components likely to be required for megaplasmid maintenance including helD (encoding a DNA helicase), repA/repB (encoding replication proteins), parAB (encoding partitioning proteins), and an AT-rich region that is predicted to be an origin of replication. After disrupting both the megaplasmid addiction and replication systems, we were able to successfully isolate strain CHC105 (ΔpHG1) that had lost the entire 452.1 kbp megaplasmid. Subsequent deletion of phcA generated CHC113 (ΔpHG1 ΔphcA).

When evaluated in MSM containing 50 mM sodium formate in microplate readers, all rationally engineered strains exhibited faster maximum growth rates than the wildtype, and several exceeded the performance of the evolved strains, especially when multiple deletions were combined in a single strain (FIGS. 1b and 1c, Table 6, Table 8).

TABLE 8
Maximum growth rates (μMax) obtained under cultivation conditions disclosed herein.

| | | Formate | Formate | Acetate | Benzoate | Fructose | Succinate |
|---|---|---|---|---|---|---|---|
| Strain | Alias | (200 μL plate) | (50 mL flask) | (900 μL plate) | (900 μL plate) | (900 μL plate) | (900 μL plate) |
| CHC001 | WT H16 | 0.080 ± 0.001 | 0.185 ± 0.005 | 0.139 ± 0.008 | 0.040 ± 0.001 | 0.125 ± 0.003 | 0.158 ± 0.003 |
| CHC076 | ΔphcA | 0.112 ± 0.008* | 0.245 ± 0.011* | 0.146 ± 0.013 | 0.039 ± 0.003 | 0.143 ± 0.002* | 0.175 ± 0.001* |
| CHC105 | ΔpHG1 | 0.096 ± 0.006* | 0.230 ± 0.006* | 0.126 ± 0.007 | 0.055 ± 0.007* | 0.136 ± 0.005* | 0.158 ± 0.012 |
| CHC113 | ΔpHG1 | 0.123 ± 0.011* | Not Tested | 0.132 ± 0.003 | 0.041 ± 0.004 | 0.149 ± 0.003* | 0.169 ± 0.003* |
| | ΔphcA | | | | | | |

Values with an asterisk indicate a statistically significant (p≤0.05) increase in μMax, compared to the wildtype grown under the same conditions.

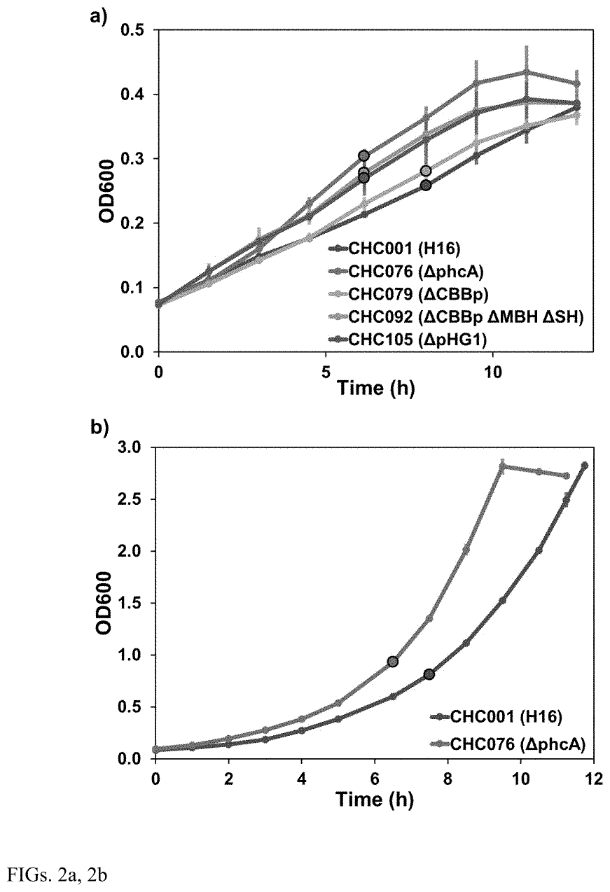

We also conducted RNA-seq to obtain the transcriptional profiles of several engineered strains and compared them to that of the wildtype when cultivated on formate or fructose in shake flasks at the 50 mL scale. Engineered strains also exhibited improved growth rates under these conditions (FIGS. 2a and 2b, Table 8). Complete raw RNA-seq results for each sample were performed, and differential expression analyses between the wildtype and engineered strains were carried out. Principal component analysis of this data confirmed that biological replicate samples clustered together, and that the majority of the variance was related to the differences in genotypes. We found that deletion of the transcriptional regulator PhcA significantly impacted the expression of hundreds of genes, while deletion of the CBBp, MBH, and SH operons had relatively minor impact on gene expression elsewhere in the genome, unless otherwise noted.

Deletion of the megaplasmid copy of the CBB operon or hydrogenase operons improves growth on formate.

When evaluated on a microplate reader, we found that strain CHC079 (ΔCBBp) displayed a 16% faster growth rate (μMax) on formate than the wildtype, although this improvement was 7% when scaled up in a shake flask (FIG. 1b, FIG. 2a, Table 6). From comparative analysis of gene expression in the rationally engineered strains, we found that deletion of the CBBp operon in strains CHC079 (ΔCBBp), CHC092 (ΔCBBp ΔMBH ΔSH), and CHC105 (ΔpHG1) in all cases led to 1.4-1.8× fold higher expression (as average transcripts per million, TPM) of the copy of the CBB operon on chromosome 2 (FIG. 2c). Surprisingly, we also found that deletion of the CBBp operon in CHC079 resulted in an 88% and 86% reduction in expression of the soluble and membrane-bound hydrogenase operons, respectively.

Deletion of the megaplasmid hydrogenase operons also improved growth. Strains CHC077 (ΔMBH) and CHC078 (ΔSH), showed 21% and 25% faster growth rates than the wildtype on formate, respectively, in microplate reader experiments (FIG. 1b, Table 6). Combining deletion of the hydrogenase and CBBp operons within a single strain CHC092 (ΔCBBp ΔMBH ΔSH) did not increase maximum growth rates further.

Deletion of the transcriptional regulator PhcA improves growth on formate and modifies expression of many genes.

When cultivated in MSM with 50 mM formate, CHC076 (ΔphcA) consistently exhibited reduced lag periods, faster growth rates, and higher maximum OD₆₀₀ values than the wildtype (FIG. 1c). The maximum growth rate of CHC076 on formate was 40% faster than the wildtype at microplate scale and 32% faster at shake flask scale, as well as 16% faster on fructose (FIGS. 2a and 2b, Table 3). Disruption of phcA was combined with deletion of the CBBp and hydrogenase operons in strain CHC099 (ΔCBBp ΔMBH ΔSH ΔphcA), resulting in a maximum growth rate 38% faster than the wildtype on formate, and similar to the performance of the evolved isolates obtained from ALE (FIG. 1, Table 6).

RNA-seq revealed that deletion of the transcriptional regulator PhcA had a widespread impact on the expression of many genes during cultivation on both formate and on fructose, particularly within major operons related to motility, surface adherence, and protein secretion. We found 59 flagellar biosynthesis and chemotaxis genes, spread between four clusters on chromosome 2, that exhibited significantly reduced expression with PhcA deleted, including a 98% reduction (as average TPM) in the principal structural flagellin gene fliC. Conversely, we found deletion of phcA led to increased expression of several gene clusters involved in the biosynthesis of type IV pili, likely used for twitching motility. We also noted a 98% reduction in expression of an flp-like pili biosynthesis operon, likely involved in surface adhesion, although two similar operons were either not affected or displayed increased expression with phcA deletion. Incidentally, we observed that ΔphcA strains had an increased propensity for flocculation under certain triggering conditions, such as upon reaching high cell densities, that may be related to changes in expression of extracellular components. We also observed that expression of one of two type VI secretion system (T6SS) clusters was reduced by 84% with deletion of phcA during growth on fructose. Deletion of phcA also reduced expression of many genes present on pHG1, including the hydrogenase operons (FIG. 2c), as well as operons involved in megaplasmid self-transmission and a PRTRC system gene cluster of unknown function.

Deletion of megaplasmid pHG1 significantly improves growth on formate.

When evaluated in microplates and shake flasks, strain CHC105 (ΔpHG1) showed, respectively, a 20% and 24% faster maximum growth rate on formate than the wildtype (FIG. 1c, Table 8). Given that the CBBp, MBH, and SH operons reside on the megaplasmid, strain CHC105 (ΔpHG1) already encompasses most of the deletions shown to improve growth on formate in our rationally engineered strains, other than disruption of phcA. To that end, we combined these modifications into a single strain, CHC113 (ΔpHG1 ΔphcA), and found that it outperformed every other engineered strain when cultivated in microplates. Under these conditions, CHC113 displayed a 54% faster maximum growth rate than the wildtype on formate (FIG. 1c, Table 8).

Rationally engineered strains exhibit improved growth on several alternate carbon sources.

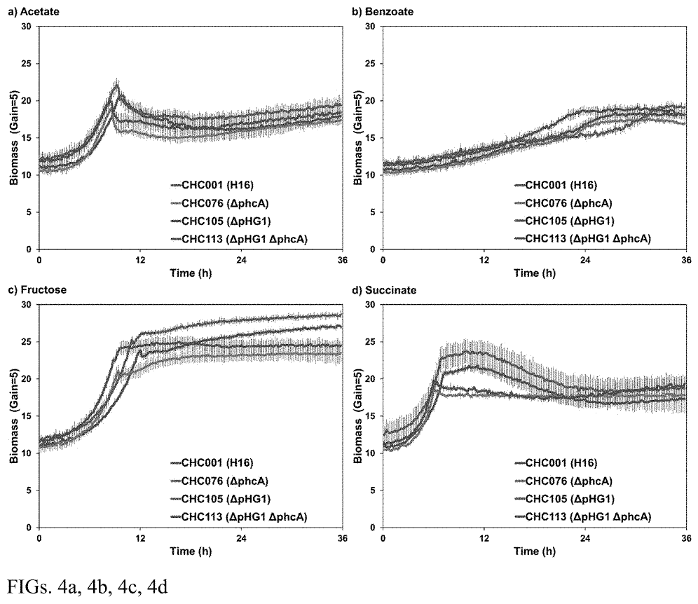

We also evaluated the impact of deleting phcA and pHG1 during growth on several other carbon sources (Table 3, FIG. 4). The rationally engineered strains grew similarly to the wildtype when cultivated on minimal media containing either acetate or benzoate as the sole source of carbon. Interestingly, CHC076 (ΔphcA) exhibited a 14% faster maximum growth rate than the wildtype on fructose, and a 11% faster growth rate on succinate (Table 3). Similarly, strain CHC105 (ΔpHG1) exhibited 9% greater μMax than H16 on fructose. Combining both deletions in strain CHC113 (ΔpHG1 ΔphcA) resulted in μMax improvements of 19% on fructose and 7% on succinate (Table 8).

Engineered strains show improved growth rates when cultivated on formate in pH-stat bioreactors.

The effect of these genetic changes was evaluated in bioreactors to determine whether their improved growth characteristics would be consistent under more industrially relevant operating conditions. Because high density growth in bioreactors is more likely to result in a nutrient limitation that could induce polyhydroxybutyrate production and confound our results, we generated PHB⁻ versions of our top-performing engineered strains by deleting the phaCAB operon, which is responsible for PHA production, to generate the strains CHC023 (ΔphaCAB), CHC122 (ΔphcA ΔphaCAB), CHC123 (ΔpHG1 ΔphaCAB), and CHC124 (ΔpHG1 ΔphcA ΔphaCAB). The performance of these strains was compared in 500 mL bioreactors under pH-stat mode where the same total amount of formic acid was fed during the cultivation.

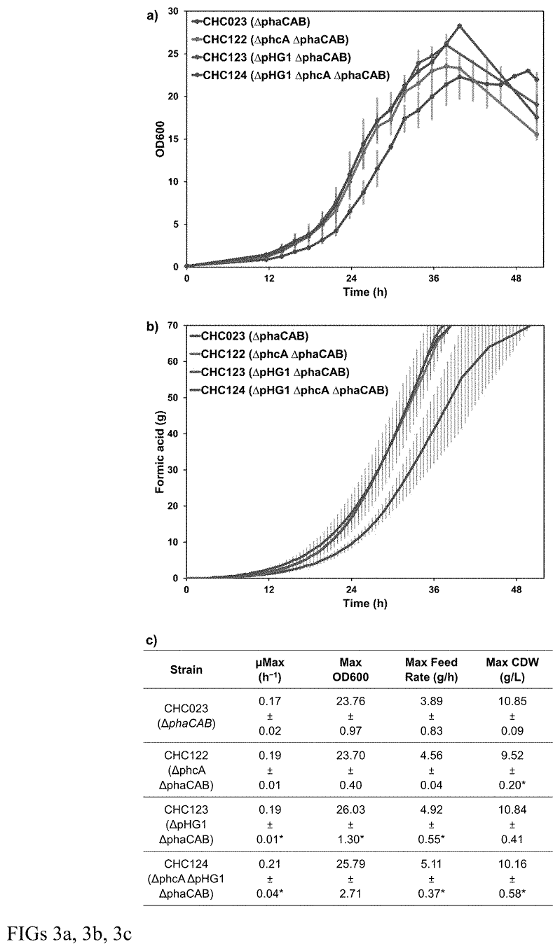

Using a pH-stat fed-batch cultivation method, the pH was controlled by the addition of a 35% (w/v) formic acid feeding solution, such that formic acid was fed at the same rate it was consumed. HPLC analysis confirmed the residual formate concentration in the bioreactors remained below 1 g/L, and that no accumulation of byproducts occurred. Consistent with results at smaller scales, we found that engineered strains grew faster and reach higher maximum OD₆₀₀ₛ than the wildtype (FIG. 3).

We evaluated the conversion of formate to cell biomass by collecting cell pellets immediately upon the exhaustion of the feed solution of each reactor. Final cell samples were confirmed to have no accumulation of PHB, due to the deletion of phaCAB. Surprisingly, despite reaching higher final OD₆₀₀ values, we found that none of the engineered strains reached higher final CDW values than the CHC023 (ΔphaCAB) control (FIGS. 3a and 3c). In fact, deletion of phcA was associated with a decrease in the final biomass; the wildtype reactors yielded 10.85±0.09 g/L cells while CHC122 (ΔphcA ΔphaCAB) yielded 9.52±0.20 g/L cells, a 12% reduction (FIG. 3c).

Nevertheless, we found that engineered strains with deletions of phcA and/or pHG1 were capable of growing and consuming formate more rapidly than the CHC023 (ΔphaCAB) control (p≤0.20). CHC123 (ΔpHG1 ΔphaCAB) reactors achieved maximum growth rates and OD₆₀₀ values each 10% higher than CHC023 (ΔphaCAB) on average (FIG. 3c). The fastest growth rate (μMax=0.21±0.04) was obtained in strain CHC124 (ΔpHG1 ΔphcA ΔphaCAB), a 24% improvement over the CHC023 (ΔphaCAB) control (μMax=0.17±0.02). The maximum growth rate we observed for CHC023 (ΔphaCAB) was similar to a previously reported value (μMax=0.18), which was, to our knowledge, the fastest reported doubling time for C. necator H16 growing on formate. The faster growth of the engineered strains was associated with more rapid formic acid feeding, which led to earlier depletion of the feed as well as faster maximum feeding rates (FIG. 3b). For example, the maximum feeding rate of the wildtype was 3.89±0.83 g/h of formic acid, while CHC124 (ΔphcA ΔpHG1 ΔphaCAB) reached a peak feeding rate of 5.11±0.37 g/h, representing a 32% increase.

### DISCUSSION

Deletion of the Megaplasmid Copy of the CBB Operon.

Whole genome sequencing of the ALE strains produced surprising results. For example, we found partial or total loss of the pHG1 copy of the CBB operon in 5 out of 6 sequenced isolates. Assuming that mutations that are most useful for improving formate utilization are more likely to appear in multiple lineages, these results suggest that there was a strong evolutionary incentive to lose the CBBp copy of the operon. This is a very surprising result, considering that the CBB cycle is essential for growth on formate, which is assimilated via oxidation to CO₂.

C. necator possesses two complete CBB operons, one located on the megaplasmid (CBBp), and another located on chromosome 2 (CBBc2), both of which contribute to growth on CO₂ and on formate. The two CBB operons are nearly identical in sequence, with two notable exceptions. First, CBBc2 contains an additional gene not found in CBBp, cbbB, that is similar in sequence to alpha subunits of the native formate dehydrogenases present in C. necator. Second, a LysR-type transcriptional regulator gene, cbbR, is present directly upstream and in opposite orientation of the CBBc2 operon, while CBBp possesses only a nonfunctional pseudogene copy of this gene, cbbR′. Expression of both CBB operons is controlled by CbbR and by an additional transcriptional regulator, RegA, which bind to DNA in the control regions upstream of each operon and act synergistically as transcriptional activators.

The intergenic control regions located between cbbR/cbbR′ and cbbLc2/cbbLp, containing promoter and ribosomal binding sequences, are also nearly identical for both operons. Without being limited by theory, this explains why expression of the CBBc2 and CBBp operons are coordinated at similar levels under autotrophic conditions. Indeed, our RNA-seq results confirmed that expression levels of both operons are relatively similar in wild-type cells grown on formate (FIG. 2c). However, we found that deletion of CBBp in our engineered strains led to significantly increased expression levels of the entire CBBc2 operon, from cbbR to cbbB (FIG. 2c). Notably, these ΔCBBp strains were constructed by deletion of the entire CBBp locus, including cbbR′ and all of the intervening control region. Given that both copies of the CBB operon contain highly homologous promoter/activator sequences, and that both operons are controlled by the same regulators, it is likely that in the absence of CBBp more CbbR and RegA are available to bind and activate expression of the chromosome 2 CBB operon, as we observed.

While this likely explains why deletion of CBBp increases expression of CBBc2, the underlying mechanism that improves growth on formate in ΔCBBp strains is less clear. Without being limited by theory, we hypothesize that the chromosomal CBB operon might be better suited for growth on formate due to the presence of the additional cbbB gene, encoding a putative formate dehydrogenase subunit. However, ΔcbbB mutants of H16 showed no significant differences compared to the wildtype when grown on formate or H₂/CO₂. Intriguingly, the cbbB gene has not been observed within the CBB operons of any other autotrophic bacteria. Further investigation is needed to determine whether CbbB is important for formatotrophy in H16. It is also possible that ALE selected for ΔCBBp mutants because deletion of the CBBp operon helps to reduce expression of the adjacent hydrogenase operons (FIG. 2c). As described below, eliminating expression of genes not required for growth on formate appears to be a valuable adaptation to formatotrophic growth, where energy is limited. Although it is not clear how expression of the CBBp and MBH/SH operons are linked, it is plausible that C. necator might have evolved regulatory mechanisms to coordinate their expression in preparation for autotrophic growth.

Deletion of the Megaplasmid Hydrogenase Operons.

The megaplasmid carries a variety of genetic clusters that enable alternative growth modes, including lithoautotrophic growth on hydrogen gas. The soluble and membrane-bound hydrogenases are large enzyme complexes that are required only when cells are grown autotrophically with H₂ as the energy source. Expression of both operons is coordinately controlled by the response regulator HoxA, which is itself controlled by a third regulatory hydrogenase that senses the presence of H₂. However, expression of the hydrogenases in C. necator is not limited to conditions where hydrogen is present; they are induced even under conditions where they are unnecessary, such as during growth on glycerol, formate, and fructose (FIG. 2c). Therefore, it is perhaps not surprising that we found one or both of these operons fully or partially deleted in 5 out of 6 of the sequenced ALE isolates. We suspect that loss of the hydrogenase operons was facilitated by the presence of the nearby 72 kb “junkyard region” of pHG1, that contains many insertion elements, transposases, integrases, and recombinases, which are known to promote spontaneous rearrangements or deletions.

During aerobic growth of C. necator on glycerol, unnecessary activity of the SH has been implicated in triggering upregulation of several cellular stress response genes, including those involved in the detoxification of reactive oxygen species (ROS). The expression of hydrogenases on formate might similarly lead to harmful ROS generation. We observed that growth on formate does trigger upregulation of C. necator stress response genes, including peroxiredoxin and superoxide dismutase. However, in the rationally engineered strains containing deletions of the MBH and SH, we found no significant reduction in expression of ROS stress response genes.

Instead, we hypothesize that deletion of the MBH and SH operons was strongly selected for during ALE because these regions of the genome are dispensable and metabolically burdensome. Indeed, the MBH and SH are biologically costly; they can account for up to 3% of the proteome by mass and both require special maturation factors to convert their inactive protein precursors to active enzymes. By not investing scarce resources into production of hydrogenase enzymes that are useless during growth on formate, it appears that ALE strains that eliminate the SH and MBH outcompete strains that retain them. The appearance of a hoxA mutation in ALE isolate GF4 supports the energy-saving hypothesis, as HoxA is an NtrC-type response regulator that is essential for activating transcription of both the SH and MBH operons. In an embodiment, elimination of superfluous hydrogenase expression is a promising strategy for improving growth on formate.

Deletion of the Transcriptional Regulator PhcA.

C. necator possesses a group of genes (H16_A3117-H16_A3120, H16_A3144) that appear to be homologous to the quorum sensing genes encoding PhcBSRQ and PhcA in Ralstonia solanacearum. In this system, PhcB produces 3-hydroxypalmitic acid methyl ester (30H-PAME) for extracellular signaling, which is detected and transduced into the cell by the two-component sensor kinase PhcS and response regulator PhcR, which (in response to cell density) collectively control expression of the LysR-type transcriptional regulator PhcA. The PhcA of R. solanacearum is responsible for activating expression of a diverse set of virulence factors, including secretion of extracellular polysaccharide I, plant cell wall-degrading enzymes, and other exoproteins. The existence of this quorum sensing module has been noted in C. necator JMP134, C. metallidurans CH34, C. necator H16, and C. taiwanensis. Of these, only C. metallidurans CH34 (formerly known as Ralstonia eutropha CH34) has been studied in detail, where it was shown that its Phc system was fully capable of complementing phcA and phcB mutant strains of Ralstonia solanacearum. The Phc (phenotype conversion) system has been investigated extensively in the plant pathogen R. solanacearum GMI1000, where phcA lies at the center of a complex yet elegant regulatory network, informed by quorum sensing, that is responsible for switching cells between specialized pathogenic and non-pathogenic growth modes. When cell density is low, such as during motile saprophytic growth in soil environments, expression of phcA is repressed by PhcR, and the virulence factors controlled by PhcA are not expressed. Conversely, as cell density increases (and 30H-PAME accumulates) during the invasion of plant tissues, quorum sensing by PhcSRQ relieves repression of phcA, and cells appropriately switch to a phenotype characterized by repression of motility and upregulation of the many virulence factors that facilitate plant colonization.

RNA-seq results on non-naturally occurring C. necator organisms generated by using methods disclosed herein demonstrate that the phcA regulatory network of C. necator shares much in common with that of R. solanacearum, including control over flagellar motility, twitching motility, and surface adherence. Interestingly, although the genetic targets of PhcA are largely the same across species, occasionally the mode of regulation is reversed. For example, deletion of phcA in C. metallidurans significantly reduces motility, consistent with our RNA-seq results in C. necator, while phcA mutants of R. solanacearum instead exhibit increased motility. It is likely that the Phc system of each species is optimized for physiological adaptation to the ecological niches that each inhabits, which can vary widely, as C. metallidurans and C. necator are not plant pathogens. Quorum sensing has never been investigated in C. necator H16, and therefore the environmental conditions in which Phc-mediated phenotypic changes might provide utility to this species remains unknown. Intriguingly, the T6SS operon we identified as under control of phcA has a high degree of synteny and homology to a system recently described in C. necator JMP134, that is capable of recruiting outer membrane vesicles (OMVs) produced by other species to gain a competitive advantage over them.

Without being bound by theory, the presence of disruptions to phcA in 4 of the 6 ALE strains suggests that this mutation was beneficial for growth on formate. We hypothesize that disruption of phcA during ALE was selected for primarily because ΔphcA cells are able to conserve energy by not generating flagella. C. necator is a peritrichous bacteria, possessing multiple flagella, each of which imposes a high energetic cost on cells, both in their initial assembly and in their ongoing operation, which is powered by the transmembrane proton motive force. For example, deletion of 70 kb of flagellar machinery in Pseudomonas putida resulted in increased ATP/NADPH availability as well as faster growth rates. Consequently, P. putida strains lacking flagellar operons, representing merely a 1.1% reduction in genome size, exhibited 40% increased titers of recombinant proteins or accumulated PHAs in metabolically engineered strains. This also could explain why our ΔphcA strains demonstrated improved growth on fructose and on succinate (Table 8), even though we did not select for growth on these carbon sources during ALE. By not allocating limited cellular resources into functions that are not necessary for growth, ΔphcA strains outcompete their less efficient comrades. Indeed, this same logic of frugal budgeting explains the purpose of the Phc quorum sensing system in R. solanacearum. In this species, PhcA induces the expression of energetically costly virulence factors only at high cell density, a condition that occurs in nature only during plant colonization, when these factors are needed. Yet, this response is maladaptive under controlled laboratory conditions, where disruption of phcA was found to increase the growth rate of R. solanacearum, as we also observed for C. necator.

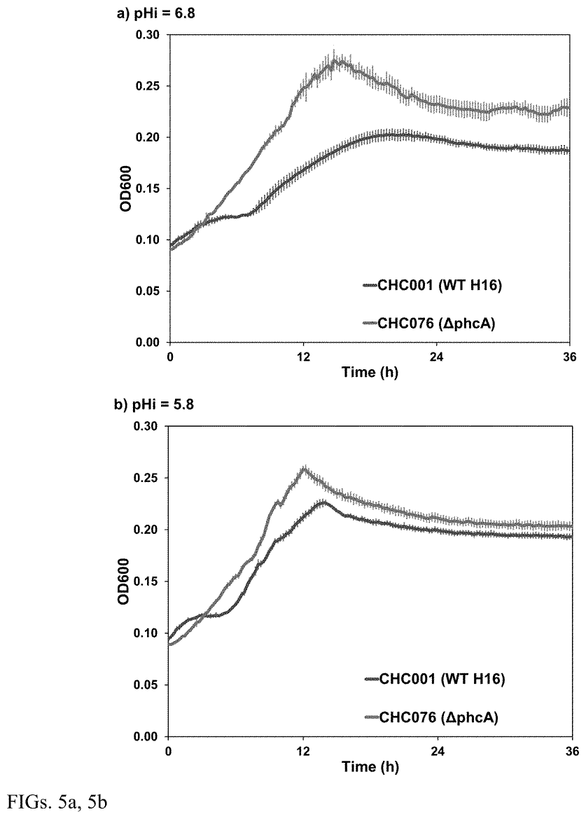

The deletion of phcA yielded growth rate improvements on formate of 40% and 32% at the microplate and shake flask scale, respectively, while yielding a more modest 12% increase over the wildtype when cultivated in bioreactors (Table 8 and FIG. 3). One critical difference in bioreactor cultivations is that the pH can be constantly controlled and was maintained at 6.7 in our experiments. Conversely, during the course of overnight growth in culture tubes during ALE, we found that consumption of sodium formate led to a substantial increase in pH, from 6.7 to 9.1. This suggests that the conditions we used in our ALE experiment may have inadvertently selected for mutations that increase tolerance to higher pH, and that deletion of phcA is more helpful for growth on formate when the pH is uncontrolled. This hypothesis is supported by the observation that by cultivating the wildtype and CHC076 on formate media with variable initial pH, much of the improvement gained from deleting phcA was eliminated by lowering the initial (and consequently, the final) pH (FIG. 5). Therefore, it is likely that deletion of phcA was selected for during ALE in part due to the improved growth of ΔphcA strains at high pH. The link between phcA deletion and improved pH tolerance may be related to optimizing usage of the transmembrane proton gradient. Proton retention is especially important for cytoplasmic pH homeostasis under alkaline conditions, where the extracellular concentration of protons is relatively low. Protons can be imported into the cell through a variety of integral membrane transporters, including the H+-coupled ATP synthase and flagellar motor machinery. Without being bound by theory, we hypothesize that elimination of the flagella is especially beneficial to ΔphcA strains at high pH because this reserves the limited proton motive force to be used for ATP synthesis and cell proliferation, rather than unnecessary motility. This is consistent with observations in E. coli, where ATP synthase expression was induced while flagellar and chemotaxis regulons were repressed in response to high pH.

Another significant difference between cultivation on plate-readers and on bioreactors is the level of aeration. Microplates depend on the oxygen transfer rate that occurs by diffusion at the surface of liquid-air interfaces, while bioreactors are highly agitated by impeller blades and further oxygenated by sparging with a continuous flow of air. Given that the 3-OH PAME signaling molecule is known to be volatile, another hypothesis is that the high rate of air exchange through bioreactors might volatilize and disperse the signaling molecule, thus preventing cells from accurately quorum sensing, and keeping PhcA somewhat repressed by PhcR under these conditions. In this case, deletion of phcA may improve the growth rate less significantly, because expression of phcA (and hence, the PhcA regulon) would be lower even in wildtype cells, due to the highly aerated growth conditions. However, this would not be the case in situations where C. necator is cultivated in closed systems, such as during autotrophic growth in pressurized bioreactors. For example, proteomic examination of H16 cultivated on H₂/CO₂ gas in sealed explosion-proof fermenters revealed changes in expression patterns of flagellar motility, chemotaxis, type IV pili, Flp-like pili, and T6SS operons that are highly suggestive of PhcA-mediated quorum sensing occurring under these conditions.

Deletion of Megaplasmid pHG1.

The 452,156 bp megaplasmid pHG1 consists primarily of genes that confer accessory functions not essential in most conditions, including large metabolic clusters related to lithoautotrophic growth, anaerobic growth by denitrification, and degradation of aromatic compounds. Interestingly, some of these functions overlap and duplicate chromosomally encoded capabilities, while others are complementary but dependent on chromosomal genes, and yet other abilities are conferred solely by pHG1. Due to the wide range of facultative metabolic activities encoded within, loss of the megaplasmid is likely to have profound consequences on growth of C. necator under certain cultivation conditions. For example, while there is substantial overlap between anoxic denitrification genes located on the chromosomes and on pHG1, only the megaplasmid contains the ribonucleotide reductase genes required for DNA synthesis under anerobic conditions. Thus, ΔpHG1 C. necator strains should be incapable of anaerobic growth. Similarly, elimination of the hydrogenase operons on pHG1 necessarily leads to loss of the ability to grow lithoautotrophically on H₂/CO₂. The megaplasmid also contains a 25 kb cluster of genes related to the degradation of aromatic compounds. These genes likely extend the catabolic capabilities of C. necator to some methylated aromatics but are not necessary for compounds degraded via the standard, and chromosomally encoded, β-ketoadipate pathway. Indeed, we found that loss of this aromatic gene cluster in ΔpHG1 strains had no significant impact on cell growth on benzoate (Table 8, FIG. 4b). Taken together, these functions implicate pHG1 as an accessory and complimentary component of the C. necator genome, that expands its metabolic versatility, and enables growth on alternate carbon and energy sources that would otherwise be inaccessible. While these functions are certainly useful under conditions where they are essential for growth, our results demonstrate that pHG1 is dispensable for aerobic growth on acetate, benzoate, succinate, formate, and fructose (Table 8, FIG. 4).

Proteomic studies of C. necator show that many of the genes required for assimilation of alternative substrates are expressed constitutively across multiple growth conditions, even when those compounds are not available. This may represent an evolutionary strategy to keep cells primed to quickly switch to alternate growth modes under rapidly changing environmental conditions, and to enable scavenging of resources as soon as they become available. While this strategy is likely advantageous in nature, maintaining this level of metabolic readiness is a suboptimal strategy for growth on a defined substrate under controlled conditions. C. necator expresses most of it annotated genes regardless of the carbon source, with about 5.4% of the proteome mass expressed from pHG1.

Previously, H16 mutants with spontaneous loss of pHG1 have been obtained by treating cells with the DNA cross-linking agent mitomycin C, which is frequently used for plasmid curing. During ALE experiments, the megaplasmid's potent toxin/antitoxin addiction system makes it unlikely to obtain mutants with total pHG1 loss. Without being bound by theory, we hypothesized, however, that pHG1 is not required or useful for growth on formate, and that a ΔpHG1 strain might outperform even our best ALE strains. To evaluate this, we developed a systematic and mutagen-free method for deleting the pHG1 megaplasmid, which has not been previously described.

We found that our ΔpHG1 engineered strains outperformed the wildtype when grown on formate, likely by eliminating the burden of replicating the megaplasmid and from the unnecessary expression of the genes it contains, especially the highly expressed hydrogenases. This energy savings benefit also extends to growth on some other carbon sources, as we observed the ΔpHG1 strain growing more rapidly on fructose (Table 8, FIG. 4c). Notably, we found that combining the ΔphcA and ΔpHG1 modifications (CHC113 and CHC124) led to increases in μMax that exceeded the growth rates on formate of either deletion individually. It follows that the energy savings from eliminating flagellar biosynthesis are complementary to, and independent from, the benefits of eliminating the megaplasmid. However, deletion of pHG1 and phcA had no significant effect on C. necator growth rates on acetate or on benzoate (Table 8, FIG. 4b). In these cases, growth is likely constrained by additional metabolic or physiologic limitations.

Understanding the Nature of Improved Growth on Formate in Engineered Strains.

To reflect on how the mutations obtained from ALE impact the metabolism of C. necator growing on formate, our most instructive results were elucidated during cultivation of our rationally engineered strains in bioreactors under pH-stat mode. By automatically feeding formic acid as quickly as it is consumed, these conditions enabled the strains to reach their maximum growth potential and demonstrated that our rationally engineered strains obtained μMax values superior to the control strain (FIG. 3c). Strains with faster doubling times should result in more rapid increases in the OD₆₀₀ as well as faster consumption of formate, due to the increased population density, as we observed (FIGS. 3a and 3b). We also noted that our evolved and engineered strains reached higher maximum OD₆₀₀ values, which could suggest that these strains are capable of more efficiently converting formate into biomass. Surprisingly, however, despite their higher OD₆₀₀ values, we found that these strains had an equal or lower final CDW than the wildtype, particularly when phcA was deleted. We hypothesize that the pHG1 and PhcA-controlled proteomes collectively account for a significant portion of cell weight and, in their absence, ΔpHG1/ΔphcA cells contain less total biomass than an equal number of wildtype cells. Thus, the increased population density we observed, as evidenced by their higher OD₆₀₀ values and faster maximum formate feeding rates, was not correlated with a proportional increase in final CDWs.

Genetic changes disclosed herein may be used to improve conversion of formate to value added products upon introduction of heterologous production pathways. Bioreactor cultivation of our engineered strains revealed improvements in growth parameters (μMax values, feeding rates, cultivation durations) that will improve the production metrics (e.g. productivity rates) of future potential bioprocesses using strains incorporating these genetic modifications.

### CONCLUSION

As disclosed herein, we developed a new platform strain of C. necator, CHC124 (ΔpHG1 ΔphcA ΔphaCAB), with improved growth characteristics. Deletion of the megaplasmid pHG1 (6.1% of the genome) and the quorum-sensing transcriptional regulator PhcA enabled maximum growth rates on formate that exceed any previously published results. These modifications also increased growth rates on fructose and on succinate, highlighting the broad utility of genome reduction as an engineering strategy. Taken together, the results disclosed herein are a demonstration that adaptive laboratory evolution and genome streamlining are powerful strategies to optimize wild-type organisms for the well-defined and highly controlled environments associated with laboratory and industrial conditions. The methods and compositions disclosed herein for the optimization of C. necator as a host for conversion of formate are applicable to other microbes under development for industrial applications.

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. The following claims are hereby incorporated into this Detailed Description, with each claim standing on its own as a separate aspect, embodiment, or configuration.

What is claimed is:

1. A non-naturally occurring Cupriavidus sp. comprising at least one genetic deletion wherein the at least one genetic deletion improves growth on formate as a sole carbon source by up to 24 percent over a naturally occurring Cupriavidus sp.

2. The non-naturally occurring Cupriavidus sp. of claim 1 wherein the Cupriavidus sp. genotype comprises ΔhoxFUYHWI ΔhypA2B2F2.

3. The non-naturally occurring Cupriavidus sp. of claim 1 wherein the Cupriavidus sp. genotype comprises ΔhoxKGZMLOQRTV ΔhypA1B1F1CDEX ΔhoxABCJ.

4. The non-naturally occurring Cupriavidus sp. of claim 1 wherein the Cupriavidus sp. genotype comprises ΔcbbR′ ΔcbbLpSpXpYpEpFpPpTpZpGpKpAp.

5. The non-naturally occurring Cupriavidus sp. of claim 1 wherein the Cupriavidus sp. genotype comprises ΔpHG1.

6. The non-naturally occurring Cupriavidus sp. of claim 1 wherein the Cupriavidus sp. genotype comprises ΔphcA.

7. The non-naturally occurring Cupriavidus sp. of claim 1 wherein the Cupriavidus sp. genotype comprises ΔpHG1 ΔphcA.

8. The non-naturally occurring Cupriavidus sp. of claim 1 wherein the Cupriavidus sp. grows in minimal salt media supplemented with 50 mM sodium formate at a growth rate of up to 2.18 times greater than a wildtype Cupriavidus sp. grown in minimal salt media supplemented with 50 mM sodium formate.

9. The non-naturally occurring Cupriavidus sp. of claim 1 wherein the Cupriavidus sp. grows in minimal salt media supplemented with 50 mM sodium formate up to a 34 percent greater optical density at 600 nm compared to a wildtype Cupriavidus sp. grown in minimal salt media supplemented with 50 mM sodium formate.

10. The non-naturally occurring Cupriavidus sp. of claim 1 wherein the Cupriavidus sp. is Cupriavidus necator.

11. A non-naturally occurring Cupriavidus sp. comprising at least one genetic deletion wherein the at least one genetic deletion improves growth on fructose as a sole carbon source by up to 19 percent over a naturally occurring Cupriavidus sp.

12. The non-naturally occurring Cupriavidus sp. of claim 11 wherein the Cupriavidus sp. genotype comprises ΔpHG1.

13. The non-naturally occurring Cupriavidus sp. of claim 11 wherein the Cupriavidus sp. genotype comprises ΔphcA.

14. The non-naturally occurring Cupriavidus sp. of claim 11 wherein the Cupriavidus sp. genotype comprises ΔpHG1 ΔphcA.

15. A non-naturally occurring Cupriavidus sp. comprising at least one genetic deletion wherein the at least one genetic deletion improves growth on succinate as the sole carbon source by up to 7 percent over a naturally occurring Cupriavidus sp.

16. The non-naturally occurring Cupriavidus sp. of claim 15 wherein the Cupriavidus sp. genotype is selected from the group consisting of ΔpHG1 ΔphcA and ΔphcA.

17. A non-naturally occurring Cupriavidus sp. comprising at least one genetic deletion wherein the at least one genetic deletion improves growth on carbon dioxide as a sole carbon source when compared to a naturally occurring Cupriavidus sp.

18. The non-naturally occurring Cupriavidus sp. of claim 17 wherein the Cupriavidus sp. genotype comprises a deletion of at least one copy of the CBB operon.

19. The non-naturally occurring Cupriavidus sp. of claim 17 wherein the Cupriavidus sp. genotype comprises a deletion of a CBB operon within a megaplasmid.

20. The non-naturally occurring Cupriavidus sp. of claim 17 wherein the Cupriavidus sp. genotype comprises a deletion of a chromosomal CBB operon.

21. A method for deleting a megaplasmid within an organism comprising deleting a gene on the megaplasmid that encodes for a toxin; and further comprising deleting a replication region of the megaplasmid.

22. The method of claim 21 wherein the organism is a Cupriavidus sp.

23. The method of claim 21 wherein the megaplasmid is pHG1.


SEQUENCE LISTING (68 sequences)

SEQ ID NO: 1 - DNA, 48 bp, synthetic construct
  source 1..48: mol_type = other DNA
aggaaacagctatgacatgattacgaattccgccggtcgcttctactc

SEQ ID NO: 2 - DNA, 54 bp, synthetic construct
  source 1..54: mol_type = other DNA
ctggttgaaccaggccggcagggtttaaacgatttgattgtctctctgccgtca

SEQ ID NO: 3 - DNA, 45 bp, synthetic construct
  source 1..45: mol_type = other DNA
acggcagagagacaatcaaatcgtttaaaccctgccggcctggtt

SEQ ID NO: 4 - DNA, 51 bp, synthetic construct
  source 1..51: mol_type = other DNA
cgttgtaaaacgacggccagtgccaagcttgcctggatgttcttttccagg

SEQ ID NO: 5 - DNA, 33 bp, synthetic construct
  source 1..33: mol_type = other DNA
gtttaaactgccttcgccggtgaaattgccaag

SEQ ID NO: 6 - DNA, 23 bp, synthetic construct
  source 1..23: mol_type = other DNA
tcaggcgctccctgcttgtttgg

SEQ ID NO: 7 - DNA, 23 bp, synthetic construct
  source 1..23: mol_type = other DNA
ggtgcagagccctacctgagtcc

SEQ ID NO: 8 - DNA, 20 bp, synthetic construct
  source 1..20: mol_type = other DNA
cgacctcgtcgtagcgcagc

SEQ ID NO: 9 - DNA, 27 bp, synthetic construct
  source 1..27: mol_type = other DNA
gattgcgtcgccgtccaccaggaaatg

SEQ ID NO: 10 - DNA, 27 bp, synthetic construct
  source 1..27: mol_type = other DNA
gaggtggaatcgtaggctgagcaggcg

SEQ ID NO: 11 - DNA, 28 bp, synthetic construct
  source 1..28: mol_type = other DNA
gctgccatgagcgaagtcacgttgatcg

SEQ ID NO: 12 - DNA, 28 bp, synthetic construct
  source 1..28: mol_type = other DNA
gcacacttggttcctgacaggccgaaac

SEQ ID NO: 13 - DNA, 28 bp, synthetic construct
  source 1..28: mol_type = other DNA
gacctccattgacgcccataatgcgctc

SEQ ID NO: 14 - DNA, 28 bp, synthetic construct
  source 1..28: mol_type = other DNA
gtagatcaccgccttgttgtaccacgcg

SEQ ID NO: 15 - DNA, 27 bp, synthetic construct
  source 1..27: mol_type = other DNA
taggcatgcgcatgggtacgaggagtc

SEQ ID NO: 16 - DNA, 30 bp, synthetic construct
  source 1..30: mol_type = other DNA
catcagcctgttatcactgcacacgctgtc

SEQ ID NO: 17 - DNA, 33 bp, synthetic construct
  source 1..33: mol_type = other DNA
agaaggtcaaagtcttcctcaacgtagatgccg

SEQ ID NO: 18 - DNA, 32 bp, synthetic construct
  source 1..32: mol_type = other DNA
acaagatctatgcctgaatccgaagacctggg

SEQ ID NO: 19 - DNA, 28 bp, synthetic construct
  source 1..28: mol_type = other DNA
ctgcgcactgaaacccagcaacttcatg

SEQ ID NO: 20 - DNA, 57 bp, synthetic construct
  source 1..57: mol_type = other DNA
aggaaacagctatgacatgattacgaattcagctgctacctcgaggctgcacaagag

SEQ ID NO: 21 - DNA, 54 bp, synthetic construct
  source 1..54: mol_type = other DNA
gacaacgcgccatgcggtcaagcatggaggttacatggcctccgcgccgacacg

SEQ ID NO: 22 - DNA, 54 bp, synthetic construct
  source 1..54: mol_type = other DNA
acgcagcgtgtcggcgcggaggccatgtaacctccatgcttgaccgcatggcgc

SEQ ID NO: 23 - DNA, 60 bp, synthetic construct
  source 1..60: mol_type = other DNA
cgacggccagtgccaagctttatttgcatagtgtttgccgactactgtttgtacatcgac

SEQ ID NO: 24 - DNA, 25 bp, synthetic construct
  source 1..25: mol_type = other DNA
cctgaacatgttctggcaccgcagc

SEQ ID NO: 25 - DNA, 27 bp, synthetic construct
  source 1..27: mol_type = other DNA
gcggatcgtcaaagatttcacgcagcc

SEQ ID NO: 26 - DNA, 29 bp, synthetic construct
  source 1..29: mol_type = other DNA
cagctatggcattgtcgagagacatggcg

SEQ ID NO: 27 - DNA, 26 bp, synthetic construct
  source 1..26: mol_type = other DNA
gcgatctgcggcagaaaggaaggtcc

SEQ ID NO: 28 - DNA, 26 bp, synthetic construct
  source 1..26: mol_type = other DNA
gctgcttcctcaaccacatcctcgcc

SEQ ID NO: 29 - DNA, 27 bp, synthetic construct
  source 1..27: mol_type = other DNA
gaatgtccagcggggacagcttcaacc

SEQ ID NO: 30 - DNA, 25 bp, synthetic construct
  source 1..25: mol_type = other DNA
cgcatccgcacgtgctagtggcttc

SEQ ID NO: 31 - DNA, 27 bp, synthetic construct
  source 1..27: mol_type = other DNA
caagctgctggaggcttcgctacttcg

SEQ ID NO: 32 - DNA, 33 bp, synthetic construct
  source 1..33: mol_type = other DNA
ctagtattgtgattggcttgccgatgactacgg

SEQ ID NO: 33 - DNA, 29 bp, synthetic construct
  source 1..29: mol_type = other DNA
ctttcccttcggtccctgaagcttgatcg

SEQ ID NO: 34 - DNA, 26 bp, synthetic construct
  source 1..26: mol_type = other DNA
gaggagatcctgcgcggcatcaagac

SEQ ID NO: 35 - DNA, 52 bp, synthetic construct
  source 1..52: mol_type = other DNA
aggaaacagctatgacatgattacgaattccgccgtcgtcgcgaactcggtc

SEQ ID NO: 36 - DNA, 56 bp, synthetic construct
  source 1..56: mol_type = other DNA
gcgagcgtgcaatcggatcggcgccaacgcggcggatcgcagtgtggcagtaagtg

SEQ ID NO: 37 - DNA, 52 bp, synthetic construct
  source 1..52: mol_type = other DNA
gtgccacttactgccacactgcgatccgccgcgttggcgccgatccgattgc

SEQ ID NO: 38 - DNA, 55 bp, synthetic construct
  source 1..55: mol_type = other DNA
cgttgtaaaacgacggccagtgccaagcttttagcggcagagtccggcgctaaac

SEQ ID NO: 39 - DNA, 23 bp, synthetic construct
  source 1..23: mol_type = other DNA
cgatgccttcctggcccaggcac

SEQ ID NO: 40 - DNA, 25 bp, synthetic construct
  source 1..25: mol_type = other DNA
ctggcgggaccattccagccatgtg

SEQ ID NO: 41 - DNA, 25 bp, synthetic construct
  source 1..25: mol_type = other DNA
gatccgccaggacgtgctcgacaag

SEQ ID NO: 42 - DNA, 26 bp, synthetic construct
  source 1..26: mol_type = other DNA
gctcatcatgccctgcatcatcgggc

SEQ ID NO: 43 - DNA, 54 bp, synthetic construct
  source 1..54: mol_type = other DNA
aggaaacagctatgacatgattacgaattcgacgatgacgaagatttctccgag

SEQ ID NO: 44 - DNA, 33 bp, synthetic construct
  source 1..33: mol_type = other DNA
ctgccactatcgtcgtcaggcgctccctgcttg

SEQ ID NO: 45 - DNA, 35 bp, synthetic construct
  source 1..35: mol_type = other DNA
gcagggagcgcctgacgacgatagtggcagcatgc

SEQ ID NO: 46 - DNA, 42 bp, synthetic construct
  source 1..42: mol_type = other DNA
tcaccggcgaaggcaggatttcatgtagttgtaggcgtcttc

SEQ ID NO: 47 - DNA, 35 bp, synthetic construct
  source 1..35: mol_type = other DNA
aactacatgaaatcctgccttcgccggtgaaattg

SEQ ID NO: 48 - DNA, 57 bp, synthetic construct
  source 1..57: mol_type = other DNA
cgttgtaaaacgacggccagtgccaagcttcaacggtatcgatcttgactacgaagc

SEQ ID NO: 49 - DNA, 740 bp, synthetic construct
  source 1..740: mol_type = other DNA
cgccggtcgcttctactcctatcggcgcgatggcgtgaccggccgcatggccagcctggtctggctggcggactgagcccgccgctgcctcactcgtccttgcccctggccgcctgcgcgcgctcggcttcagccttgcgtcggcggcggccgggcgtgcccatgatgtagagcaccagcgccaccggcgccatgccatacatcaggaaggtggcaacgcctgccaccacgttgtgctcggtgatcgccatcatcagcgccacgtagagccagccaatggccacgatgtacatcaaaaattcatccttctcgcctatgctctggggcctcggcagatgcgagcgctgcataccgtccggtaggtcgggaagcgtgcagtgccgaggcggattcccgcattgacagcgcgtgcgttgcaaggcaacaatggactcaaatgtctcggaatcgctgacgattcccaggtttctccggcaagcatagcgcatggcgtctccatgcgagaatgtcgcgcttgccggataaaaggggagccgctatcggaatggacgcaagccacggccgcagcaggtgcggtcgagggcttccagccagttccagggcagatgtgccggcagaccctcccgctttgggggaggcgcaagccgggtccattcggatagcatctccccatgcaaagtgccggccagggcaatgcccggagccggttcgaatagtgacggcagagagacaatcaaatc

SEQ ID NO: 50 - DNA, 750 bp, synthetic construct
  source 1..750: mol_type = other DNA
cctgccggcctggttcaaccagtcggcagccggcgctggcgcccgcgtattgcggtgcagccagcgcggcgcacaaggcggcgggcgtttcgtttcgccgcccgtttcgcgggccgtcaaggcccgcgaatcgtttctgcccgcgcggcattcctcgctttttgcgccaattcaccgggttttccttaagccccgtcgcttttcttagtgccttgttgggcatagaatcagggcagcggcgcagccagcaccatgttcgtgcagcgcggccctcgcgggggcgaggctgcaggccgccacgcgcagccatgcgcgaacgggccaccagatggccggcacgacaacaagcagatggcgcgggcgataccgatttgcgcactgcaccccatgcggtgcagcagcgcgcaaacagcgatgacacaaggacagagcaccgatggccacgaccaaaaaaggcgcagagcgactgatcaaaaagtatccgaaccgtaggctctacgacacccagaccagcacctacatcaccctggccgacgtcaagcagctggtcatggattcagaagaattcaaggtcgtcgacgccaagtctggtgacgaactgacccgcagcatcttgctgcagatcatcctggaagaagaaacgggcggcgtgccgatgttctccagcgcgatgctgtcgcagatcatccgcttctacggccatgccatgcagggcatgatgggcacctacctggaaaagaacatccaggc

SEQ ID NO: 51 - DNA, 1021 bp, synthetic construct
  source 1..1021: mol_type = other DNA
gacgatgacgaagatttctccgagcagtgtttttcgtccggcatacttttttcatgtcgccaagcaagctgagcgattcctgaaggctcaaaccaatggcactggcattccacacgttgaccgagagcttctcgaggggataaaggtcttttgtcctggctctacggagcagcaattacttgcggaaatcctcgacactctcgacaccgccatctacgaaactgaagcgatcatcgccaagctcaaggcggtcaagcaaggcctgctgcatgacctcttgacgcgcggcatcgacgccaacggcgaattgcgcccacctcaggccgaggcaccgcatctctacgagtcgtcaccgttgggttggattccgaatgagtggggtcttgctcctacagcaactcgctgccatctgataaccaaaggcactacccctgcggctaatgagatgtggcagggtggcgcgggaattaggtttctgcgagtcgataatctttctttcgatggacaactggatctagatgcaagcacgtttcgagttagccttgccacgcacaaaggttttctggctcgttcaagatgccttgaaggtgatgtgctgacgaacatcgttggcccacctctagggaaactggggcttgttaccaaagaaattggtgaggtcaatattaatcaagcaattgcgttatttcgaccaaccgaacaactactgccaaagttcctattaatctggcttagtagctcaatctcgcagtcttggctgaggaaccgagccaagcagacgtcgggacaagtgaatctgaccctcgctctatgccaggagcttcctctacctcggatgacgatcaatgagcaacaggcaatcgttgaccgagttgatgccgcgcaggaacaaatctggtgtgaggaggaactgatccgaaagatgcgacttgagaaatctggccttatggatgacctcctcaccggccgcgtccgcgtcaagccgcagctggcggaaaccaaacaagcagggagcgcctga

SEQ ID NO: 52 - DNA, 1047 bp, synthetic construct
  source 1..1047: mol_type = other DNA
tgccttcgccggtgaaattgccaagccttcagatcggtgacctccggttcacgctccagcggagcgcgcgccgcagaactatgcagatcaccgtggagcgcagtggcgacttgatgctctgcgcaccgccggaggtggacgaggccgcgctgcgagcattcgtgctggagaagcgcttctggatctacaccaagctggccgagaaggaccgcttgcagcgccaggttccgcgcaaggaattcgtcggaggcgagggattcttgtatctcggccgcagccatcggctgaaggtggtcgatgaacagaatgtgccactgaagttgaatggaggccgcttttgtctgcgccgtgacgccctacccgccgcgcgcgagcatttcatccgctggtacggcgagcgtgccaaggcctggctttcggggcgtgtagctgactaccagtcgcgaatggaggtgacgcctgccggcgtcaaggtgcaggaccttggatatcgctggggttcgtgtggcaagggcgactggctgtacttccactggaaggcaatcctgctgccggcgcgcatcgctgagtatgtcgtggtgcatgagattgcccatctgcatgagccgcaccacacgcctgcgttctggcttcgagtggagcgtgccatgccggactatgcgcaacgcaaggcctggctggccgagcatggaatcgatgttgaaggaatctaaagaacgatggctgactatttcaccagtgactacttcaagctgctgaacaagtggaaggggcagaagcgtgacgagtccaaccccgagcagaaccgcgcttatgaagatctgaagaaggcctacgaggtgacggaggcgtgggcggacaaggttaaggccgagttgttccctgtcgggcgcgtcgagattcgtaagcgcccgaccaaccagggcaacaactttgccagctacaactgggccaaaatctacccttcatctgaggcgccgaaagagttggcttacacagttggcatcggcgccgatgacggcttcgtagtcaagatcgataccgttg

SEQ ID NO: 53 - DNA, 750 bp, synthetic construct
  source 1..750: mol_type = other DNA
acgacttcgccaaggaacaggtcgtaggtctgctgcgtggccggctccggcagcagccggcattccagccaggccgcgcagccctccagcagcggcgcgcccaccgccgtgccggcaaaggtgccaaggccgtaggcgtcgaacttgtcggtgccttcctgctccatcagcgccaggcccgagctggagcccagtgcctcggtcaggtcgacctggctgacggtggggacctgcaacacgaactcgccgctgtcttccagcaggtgccgggtccaggtgctcttgtccagcaccactgccaccttgggcggggcgaagtcgagcggcatggcccaggcggcggccatgatattgcgcttgccgccggcggcggcgctgaccagcacagtggggccgtggttcagcaagcggtaggctttcgggagtgatacgggcaggcggaaatgttcaggcatgatggccgggatgagccgtcagaaaagaatgataaaaatgggaacggcggacccactatacccggatgtacgagtgcatgttgcggcgcgggaaatgttcacatatgcggtcaattgtggaaaaagagcgcaatttttcagaaatatggcgtagacggccatttcagaaatgccgaatttgctttccgagcttgttttttcctcttacactattaagacgccgttgaaatctgatgtgcagccagtgcaagtggtggggccatctagctaagaataatctgaccgaggcctgatc

SEQ ID NO: 54 - DNA, 750 bp, synthetic construct
  source 1..750: mol_type = other DNA
gcagcatcctgcggcgagcagcccaaacaaaaaaccggcgcctggcgccggttttttgttgcccgtctgcgctccgcggtggagcgtgcaggcttatcgtttgggtctgtggggacagtctgttttggtgcaattgccgtacagcgacagtgcatgttcctgcagcgtaaagccgcgctcgcgcgcgatgctttgctggcggtgctcgatttcagagtcgaagaactcctcgacgcggccgcagtcgaggcacaccaggtggtcatggtgcttgccttcgttgagttcgaaaatcgccttgccggattcaaagttgttgcgcgagagcaggcccgcctgctcgaactgggtcagcacgcggtagacggtggccaggccgatgtccatatgctcgttcagcaggatacggtagacgtcttccgcgctcaggtgccgctgctcactggtctgaaaaatttcaagaatcttcagcctgggcacggtcgccttcaggccgatgttcttgaggtccgccggactcggcatgtgggtgactccctagagtacaatgactggatagttgaatcataagggttttggcagcaaaagtcgctcgcggtagtgatgtcccaggtcgacgtcacgcgcccggcacggttgccgtcccatgtggttgtcgcggcaggcgcaacaaggcgtgtgtggtgcaatcggcgcatattgcgccgtttttgtgcccgctttgcggtaccgtgccgcggtacttttctt

SEQ ID NO: 55 - DNA, 750 bp, synthetic construct
  source 1..750: mol_type = other DNA
gagctatgtcgcaccttcctgctggaacacacggcggaatatttggaacgggaatacggcgggctgctgcccggcgggcttgtggcctaggggattcggcaagtcggggatcctggtagatggcgtcggcctgcgcatgtgtcatggcgccggtggcgagataagcatccggcaccacggcagttgggcttgcgggctggtctgcagtagctgcgtggcattgctcggacgacagacccgctcctgccgcgcatgcagcaggggtacaggaaggctgcgagcagcgccggctcattgcctttccgttgggcggggcgagacgccgggggcgggggctcatccgagttcaacgccgatcactgaacttccttctgatgcattcaagcgaaaacccagtgagcatctggcgtcggctagcgccaggcgacggtccacttcatgacggatgaaatattgtcaaatcaggatccggtgtcctgcgttgtaggttcgccgaatagggcgctgtcgggcggacgcacgaacctgcgtcacagatgctcatacatgccttctcggtatcaatctttttctaaacaagccatccaactcaggatggtagcgggggttttccccaggtcttcggattcaggcatagatcttgtttcaactatgtcgccaagccagcattcgtgcgcgagggcggtatcgctccccggttggcgcatcgcgacgaatgccaataccaatacagaaattaggagacaggtt

SEQ ID NO: 56 - DNA, 750 bp, synthetic construct
  source 1..750: mol_type = other DNA
tcggttgccggggcccggctccgcccgtgttccggggaacgcctgttcgaaattggcggaggcaggaggctgatggcctgatttccctgctgcaccaggctagaaagcgctgctccggctatttagactcccatggaacatggtattgccatctggatatgggcatgtcaccaatgcgatgatcatgcaaacctgctttgcagtcctcacgtacggacttgcgcagcagataccgctatttcgggaatagcataagcgaaccaagacctgagagtgagcttctgccgcattcgccaggagttggctcgcaggcgcggaaattgcgttacggtgcagtcgagccttactggcaaaagccgcggatgacagcggcgtcggaaccgagacaggagacttccagcatgttccaattgctcgctggcgtacgcatgaattctactggccgcccgcgggccaagatcatcttgctctacgcgctgctgattgcattcaatatcggcgcctggctctgcgcgctcgccgcgtttcgcgatcatccggtgctgctcggcaccgcactgctggcctacggccttgggttgcgccacgcggtagacgcagatcatctcgcggcaatcgacaatgtcacccgcaagttgatgcaggacggcaggcggcccatcacagctgggctttggttctcgcttggccattcaagtgtggtagtgcttgcttcggtgctgatcgctgtcatggcgaccacgctccagg

SEQ ID NO: 57 - DNA, 750 bp, synthetic construct
  source 1..750: mol_type = other DNA
ggcacttggggcaatgccggttgcggcaggtaatggagagcaacccattacctgccggcatcgcgcctgcccgaagtgccagtcgctcgcccgcgcgcaatggctcgaacaccggcaggctgagctgctgcccgaggtcgagtatttccatgtggtcttcacggtgcccgaccccatcgcggcgctcgcctatcaaaacaagaatctctatgacatcctgttccgcaccagcgccgaaaccctgcgcacgatcgccgccgatccgaaacacctgggcgccgagatcggcggccagacctcatcgggtcctgctcataggttcgtagccgcgatcgccaaccaaaaaaaccctctcctgcgggaaatccgcacgctacgttctgtgggaaccggaggcgggtgactgcctccggtcacccggtgctcggggtgcgattccccgggtctacttaccaaatcggccgcgcacccaatgagaggcgctggcacaagcttgcacagacttgcccgccaagcggaagcagccttgccacatcggccgacccaatggcaatgccgctgccacccgccggatggccgttctggaaacggcttgagcgacgtcaagaatttcctttctcgacaagcacttagccgggcctcctggtggtttcccttaggccctgcgaaattggcgcacatcctgcgttccacctgcgcatcgaagtgacgcaccaagcaaggggcgaacattagtaaggaggagacaac

SEQ ID NO: 58 - DNA, 750 bp, synthetic construct
  source 1..750: mol_type = other DNA
cgagagggtagaacatgtgcctggccatacctgcacgcatcgcgaaaaaatttgacaacgacatggccctcatcgacctgggcggcgtggggagtggccaaaaacggggggcaaatccgtcaggaaaggggtctattgtgtactgagactaccggagaccgccatgcgcatctcgatccaagcctgtattgagcgggcgggcgaacagccctctaaggtgattgaagttgcggtgatcgagcgcaatgccgatgtcgctccggcctcaggactgggcctgttcattcgcgagtcacaagagatcctgcgacagcttcagactgtggtcttgaccgagcaggtggaccagttcatccggattaccggtcgctgtcaactgtgcggaggcaggcttgtcatcaaggacacaaaatccttggtctatcgcaccgcttttggcaaggcgaggctgcgaagcccgcgcttttactcttactgcagcgcatgcggttactgctcaagtaacaagggcacgctttccccgctggcacaggcgttaccagaacgcgtacatccccagtggacctggctgcagtgccgatatgcaagcgtgatgtcttatcgtttggcacagatctttctacgcgacgcgtttgccggcggacgggaactcccatgctcgagcgttaagttgaatgtaggccgggtcgggcagcggctggagcaagaggcgcaacgtgcaacgatggtgatgtcggctgtgaccgcgcc

SEQ ID NO: 59 - DNA, 750 bp, synthetic construct
  source 1..750: mol_type = other DNA
agttggatggcttgtttagaaaaagattgataccgagaaggcatgtatgagcatctgtgacgcaggttcgtgcgtccgcccgacagcgccctattcggcgaacctacaacgcaggacaccggatcctgatttgacaatatttcatccgtcatgaagtggaccgtcgcctggcgctagccgacgccagatgctcactgggttttcgcttgaatgcatcagaaggaagttcagtgatcggcgttgaactcggatgagcccccgcccccggcgtctcgccccgcccaacggaaaggcaatgagccggcgctgctcgcagccttcctgtacccctgctgcatgcgcggcaggagcgggtctgtcgtccgagcaatgccacgcagctactgcagaccagcccgcaagcccaactgccgtggtgccggatgcttatctcgccaccggcgccatgacacatgcgcaggccgacgccatctaccaggatccccgacttgccgaatcccctaggccacaagcccgccgggcagcagcccgccgtattcccgttccaaatattccgccgtgtgttccagcaggaaggtgcgacatagctcgctggcgggcgacagccgcttgctagccatgtgcacgacatgccagacacgctcaattggcgtgcctgccgcatcgagcagcgcgatctcccggtgtgtgcaattccagcgacagcgtgtgcagtgataacaggctgatgcccatgccggccatcaccgc

SEQ ID NO: 60 - DNA, 750 bp, synthetic construct
  source 1..750: mol_type = other DNA
gcgagacgtagtcagcgaacatgccatccggccccttgctcatgctggaatcaccgagagtgtggtccgcagctggggtgcgctcatggcaggtgccttgtcggcatctgtcaccggtagcgtgcccggccgtgcaacgcactggcggagtagcatggacagcttggcttgcagcatttcggcggtgccgcatagcgaggaggcaaggggcggtggcgtggtacatgggatcggctcgggtgctatggctgctccaagtgcagggaggcatggcgcccggctggcgctgcacaatcgggaaccgcccgctgctaggcgtatgcggacaggcgattctctcgcgccaaacgtggcttctagcggcattctggtagccggctctcgcggtcgtcgggcttttcagaatctgtcttactaaccttctcgaaagtattgtcatgtcatgagacaatacgggaatgaaatgcaaacggaactcggacggtcgagcgatcgatcatcatgcccttcaggtgatgcgccaacaggcgatcaaagcagttcgtgagggtcaaacggcgcaaagcgtggcggcggcgctgggcgtgaatgtgcgaagcgtcttcaggtggcttgccgattatgctagcggtggccagcgtgcgctgctcgccaaaccgatcccggggcgtccgtccaaagtcagcggcgacgagatgcgctggcttgcccaagcggtgcgagacaacacaccgcagcaatacaagttcg

SEQ ID NO: 61 - DNA, 750 bp, synthetic construct
  source 1..750: mol_type = other DNA
agctgctacctcgaggctgcacaagagattcgagccgattgccataacccactgcgacaggtcacctggtaggggctcagcgtcgcgccaagtgtggcagccgtggcgtcggccagcgaagaaagcgccccgcggcggtgcagatttccggcgggttgccgagaaaggaggctcaaatgtcctcacaggaaaacagcggccatgtcgaacaaggcaatggcaaccgtgtcgaagctggcgctagcggggctgcggcatggcgccggtgcggtagccggagcccggcgcggcgccaagcagatacgcaggcttctgccggcagactccttcgcgcgttcggctgcgcagcgccgctgcgccgtcagtcgtcacgcgggcaaatttcatttgttggcagcgatggcgagcaacgctcgagggagcgatgtagatacgcgttgaaacatggatctcttatgtttatacttgtatcaacattgtttggaggcatctattatgcgaaaaagcgcaaccctgacgattcaaaagtggggcaacagcttggcggttcgaatccccactgcggtggctcgttctgcacatttcgccgagggccaggaagtggaggtatccgtcgatgagattggcgtaactgttcgaccagttggtcgtcgtgccctcactctcgcggaaaagcttgctctgtttgaccccatcaagcacggcggcgaagctatggccacgcagcgtgtcggcgcggaggccatgtaa

SEQ ID NO: 62 - DNA, 750 bp, synthetic construct
  source 1..750: mol_type = other DNA
cctccatgcttgaccgcatggcgcgttgtcttggcgaacacgccgagccgtcgcaagctggtcactgccagcgcacacatgcggctcgactggatgaagccagccacggtagacgtagacgtgatcagcacatcaacctgaccaccgagcagatcattgatggccgacccggcgcccttgtacggcacgtgctgcagcggcacgctgctgttcttgtcgagcacaacgcctatcaggtgcaacagcgtgccgatgccgggcgtggcgtaggtgatcttctgcagttgtgccttggcctgtgtggccagcgccgggcagtgggcgggactcgtcgcgccgcgcggtccgcgtgccgaagtcactgcgccgcccaaggttcgcgctgcggaactgggcgccggcctgcgtgagcgtcaatgctggcggcggcccccccggcagcatggcgcgccgccaaccccatgttgaggttgtgtcccggcgctactggtcaacgcgcacaatcaacctcacctggcgcacaacctacctcaccgatccctgccggctgtttccttccagcctttgtccagcttgggagataagacatatgcacaggtcacgcacaatacatctcaccttaggggcatcaacacaacaaacctcaccttcttggggcggcttcggatgcggtgccgttcatcaggcatcgtgtccgccgtaacggggatgtcgatgtacaaacagtagtcggcaaacactatgcaaata

SEQ ID NO: 63 - DNA, 750 bp, synthetic construct
  source 1..750: mol_type = other DNA
cgaacttgtattgctgcggtgtgttgtctcgcaccgcttgggcaagccagcgcatctcgtcgccgctgactttggacggacgccccgggatcggtttggcgagcagcgcacgctggccaccgctagcataatcggcaagccacctgaagacgcttcgcacattcacgcccagcgccgccgccacgctttgcgccgtttgaccctcacgaactgctttgatcgcctgttggcgcatcacctgaagggcatgatgatcgatcgctcgaccgtccgagttccgtttgcatttcattcccgtattgtctcatgacatgacaatactttcgagaaggttagtaagacagattctgaaaagcccgacgaccgcgagagccggctaccagaatgccgctagaagccacgtttggcgcgagagaatcgcctgtccgcatacgcctagcagcgggcggttcccgattgtgcagcgccagccgggcgccatgcctccctgcacttggagcagccatagcacccgagccgatcccatgtaccacgccaccgccccttgcctcctcgctatgcggcaccgccgaaatgctgcaagccaagctgtccatgctactccgccagtgcgttgcacggccgggcacgctaccggtgacagatgccgacaaggcacctgccatgagcgcaccccagctgcggaccacactctcggtgattccagcatgagcaaggggccggatggcatgttcgctgactacgtctcgc

SEQ ID NO: 64 - DNA, 750 bp, synthetic construct; identical to SEQ ID NO: 58
  source 1..750: mol_type = other DNA

SEQ ID NO: 65 - DNA, 750 bp, synthetic construct
  source 1..750: mol_type = other DNA
cgccgtcgtcgcgaactcggtctgctgatcctcaatcgcgagcttgccggcgcggcagtcgttcccgtggcgctgaagctcaatcacacgcgcctgcgcccgctcttcgagcagtggtggccatacatgaaccgcatgtcgctgaacctgcagcgcttcggggcggctactttctcgcgcaccgaactgtcgacgctcgagaactacctcgaacgcgagctctcgaagatcgaggactatgtggacgaacagctgcgtgtggcgaaagcctaccgcgagcaacgggaacaggagatgcgagcgagaggcgagatcgtgttcgtcccgacgatccagcgcccgtcccttgcgctcgaggtccaggcctactcgcgcttttccgtgcgtgccctgcaggttctgatcaagttcgatcaaaccatggaccagttcgactttatggtctggaacggcatccgtgaccagagcgacgtcaacgatgaagtcacgcgcttcctacgcaagttccagccgctgggcctgcgcagctacaccactcacctgaggttgatgacgacggtgcgctgtatttgaccacaggaattgatgtgacatggtgtcacttggtgtgagctggtgagaccacgtgggacatgatctcacttccgaacgaatcaataggcgtcgacatctgccccgaggtaccacttcggggcacttgctgttttgaaccggcaccaagtgccacttactgccacactgcgatccgcc

SEQ ID NO: 66 - DNA, 750 bp, synthetic construct
  source 1..750: mol_type = other DNA
gcgttggcgccgatccgattgcacgctcgcctgctgacctacctctctgaggtgagcaacatgaccgctccgctcctgttggttgagatcgtctcggaacggcgcgttaacaaaagcactatcgcctgcccaaacgactcacggctttgctgccaacaggagaccccccagatgaataacgcttccgagtaagtcactggaaggcttcgtgttccgactcggaacaccgaagctcaaaggtctgaagcagcatcacccgactaaggcgggtcgtgcgggtgtccgtgttctgacttggaacgcagctgagtttgtcccggcgatacctcgggcgagcgcattatgggcgtcaatggaggtcgcttacggggacccttgtagggcaggcactgcaaacggacagcagcaatagctttgcggcgcaagatcgcgctgttatggagcgctgacggccgaggacacgtcgcctgaaagagtgcgacatgataaaggcgggcgccgagcgccggctctcatagggggaccgagtttggcatccgtggccacacctctcaatgagttgccttacgtcacccagagcttgcatgccccggattcgtctcgtctcaacttgcgacataccctcgcgggatcccgcgacacgatggcgcaaggaccgacagcaaggtcacaaagtttgtacgaatcctagttgcaaggcctccgtaagccctgtgcgcggacttgtttagcgccggactctgccgctaa

SEQ ID NO: 67 - DNA, 750 bp, synthetic construct; identical to SEQ ID NO: 53
  source 1..750: mol_type = other DNA

SEQ ID NO: 68 - DNA, 750 bp, synthetic construct; identical to SEQ ID NO: 54
  source 1..750: mol_type = other DNA